(19) United States
(12) Patent Application Publication
(10) Pub. No.: US 2024/0041954 A1
(43) Pub. Date: Feb. 8, 2024

(54) USE OF LACTIC ACID BACTERIA TO INHIBIT METHANOGEN GROWTH OR REDUCE METHANE EMISSIONS

(71) Applicants: Fonterra Co-Operative Group Limited, Auckland (NZ); Agresearch Limited, Hamilton (NZ)

(72) Inventors: Graeme Trevor Attwood, Hamilton (NZ); Laureen Crouzet, Hamilton (NZ); Shalome Anitta Bassett, Auckland (NZ); James William Dekker, Auckland (NZ); Jeremy Paul Hill, Auckland (NZ); Wayne Young, Hamilton (NZ)

(21) Appl. No.: 18/259,014

(22) PCT Filed: Dec. 22, 2021
(86) PCT No.: PCT/IB2021/062162

§ 371 Date: Jun. 22, 2023

(30) Foreign Application Priority Data
Dec. 22, 2020 (AU) 2020904789

(51) Int. Cl. A61K 35/747; C12N 1/20; A61P 31/04; A61P 1/14; A23K 10/18; A23K 50/30; A23K 50/60

(52) U.S. Cl. CPC A61K 35/747; C12N 1/205; A61P 31/04; A61P 1/14; A23K 10/18; A23K 50/30; A23K 50/60; C12R 2001/225

(57) ABSTRACT

This invention relates to use of a strain of lactic acid bacteria for inhibiting the growth of methane-producing bacteria and/or archaea in the gastrointestinal tract of monogastric animals, reducing the ability of the microbiome of the gastrointestinal tract to produce methane, reducing methane production or emissions, and/or for improving feed efficiency, and/or body weight or body composition of a monogastric animal.

### TECHNICAL FIELD

This invention relates to use of a strain of lactic acid bacteria for inhibiting the growth of methane-producing bacteria and/or archaea in the gastrointestinal tract of monogastric animals, reducing the ability of the microbiome of the gastrointestinal tract to produce methane, reducing methane production, and/or for improving feed efficiency, and/or body weight or body composition of a monogastric animal.

### BACKGROUND

Methane is a potent greenhouse gas, absorbing infrared radiation much more efficiently than CO₂ and having a warming potential ˜86 times larger than its mass equivalent of CO₂ on a 20-year timescale (IPCC, 2014). While methane is a relatively low proportion of anthropogenic greenhouse gas emissions, it nevertheless is a significant contributor to climate change.

A major source of methane emissions is the fermentation of organic matter by methanogenic bacteria and archaea. One prevalent source of anthropogenic methane emissions is in agriculture, such as where methane is produced by fermentation of livestock manure. For example, methane emissions from pigs account for ˜10% of the total methane production from livestock in China (Mi et al., 2019). In addition, not only does methanogenesis result in greenhouse gas emissions, but it is also energetically wasteful. It has long been recognised that methane production in livestock impacts the efficiency with which these animals convert feed into metabolic energy. This decrease in efficiency results because methane represents a caloric loss to the animal, and represents ˜0.1-3.3% of digestive energy loss in pigs (Mi et al., 2019).

Thus, there remains a need for methods for inhibiting the growth of methane-producing bacteria and/or archaea in the gastrointestinal tract of monogastric animals, reducing the ability of the microbiome of the gastrointestinal tract to produce methane, and/or for reducing methane emissions by a monogastric animal. Methods for improving feed efficiency, increasing body weight and/or improving body composition of a monogastric animal are also desirable.

It is an object of this invention to go some way towards achieving one or more of these desiderata, or at least to offer the public a useful choice.

### SUMMARY OF THE INVENTION

In a first aspect the invention provides a method for inhibiting the growth of methane-producing bacteria and/or archaea in the gastrointestinal tract of monogastric animals, or for reducing the ability of the microbiome of the gastrointestinal tract to produce methane, wherein the method comprises administering to a monogastric animal an effective amount of Lacticaseibacillus rhamnosus strain HN001, AGAL deposit number NM97/09514 dated 18 Aug. 1997, or a derivative thereof.

In a second aspect the invention provides a method for reducing methane production by a monogastric animal, wherein the method comprises administering to the animal an effective amount of Lacticaseibacillus rhamnosus strain HN001, AGAL deposit number NM97/09514 dated 18 Aug. 1997, or a derivative thereof.

In a third aspect the invention provides a method for increasing feed efficiency in a monogastric animal, wherein the method comprises administering to the animal an effective amount of Lacticaseibacillus rhamnosus strain HN001, AGAL deposit number NM97/09514 dated 18 Aug. 1997, or a derivative thereof.

In some embodiments, the method inhibits the growth of hydrogenotrophic methanogens in the gastrointestinal of the animal. In one embodiment, the method inhibits the growth of a methanogen from the genus Methanobrevibacter in the gastrointestinal tract of the animal.

In some embodiments, the method inhibits the growth of hydrogenotrophic methanogens in the caecum or colorectum of the animal. In one embodiment, the method inhibits the growth of a methanogen from the genus Methanobrevibacter in the caecum or colorectum of the animal.

In some embodiments, the L. rhamnosus HN001 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet or bolus.

In some embodiments, the L. rhamnosus HN001 or derivative thereof is administered in drinking water, milk, milk powder, milk replacement, milk fortifier, whey, whey powder, a feed pellet, corn, soybean, forage, grain, distiller's grain, sprouted grain, legumes, vitamins, amino acids, minerals, fibre, fodder, grass, hay, silage, kernel, leaves, meal, solubles, slurries, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushed limestone.

In some embodiments, the method comprises administering to the animal L. rhamnosus HN001 in an amount from 10⁴ to 10¹³ colony forming units per day.

In some embodiments, the method comprises administering to the animal L. rhamnosus HN001 in an amount from 10⁸ to 10¹² colony forming units per day.

In some embodiments, the derivative of L. rhamnosus HN001 is a cell lysate of the strain, a cell suspension of the strain, a metabolite of the strain, a culture supernatant of the strain, or killed L. rhamnosus HN001.

In some embodiments, the method comprises further administering at least one additional microorganism of a different species or strain, a vaccine that inhibits methanogens or methanogenesis, and/or a natural or chemically-synthesised inhibitor of methanogenesis and/or methanogen inhibitor. An example of a useful inhibitor of methanogenesis is bromoform, which works by inhibiting the efficiency of the methyltransferase enzyme by reacting with the reduced vitamin B12 cofactor required for the penultimate step of methanogenesis.

In one embodiment, the method comprises further administering at least one microorganism of a different species or strain, a vaccine that inhibits methanogens or methanogenesis, and/or a natural or chemically-synthesised inhibitor of methanogenesis and/or methanogen inhibitor (such as bromoform) that targets a methanogen that is not Methanobrevibacter, for example, a methylotrophic methanogen such as a methanogen from the genus Methanosphaera or the order Methanomassiliicoccale.

In some embodiments, the L. rhamnosus HN001 or derivative thereof is administered separately, simultaneously or sequentially with one or more agents selected from one or more prebiotics, one or more probiotics, one or most postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

In some embodiments, the method increases the body weight and/or improves body composition, such as altering the muscle to fat ratio, of the monogastric animal. In some embodiments, the method reduces the body mass index (BMI) and/or increases the muscle to fat ratio of the monogastric animal.

In some embodiments, the monogastric animal is a human, pig, cat, dog, horse, donkey, rabbit, or poultry. In some embodiments, the monogastric animal is a pig. In some embodiments, the monogastric animal is a chicken, duck, goose or turkey.

In some embodiments, the monogastric animal is a pre-weaning animal, for example a piglet or a foal.

In a further aspect, the invention provides a method for enhancing the growth and/or productivity in a monogastric animal, wherein the method comprises administering to a monogastric animal an effective amount of Lacticaseibacillus rhamnosus strain HN001, AGAL deposit number NM97/09514 dated 18 Aug. 1997, or a derivative thereof.

In a further aspect, the invention provides a method for improving the body weight and/or body composition of monogastric animal, wherein the method comprises administering to a monogastric animal an effective amount of Lacticaseibacillus rhamnosus strain HN001, AGAL deposit number NM97/09514 dated 18 Aug. 1997, or a derivative thereof.

In a further aspect, the invention provides use of Lacticaseibacillus rhamnosus strain HN001, AGAL deposit number NM97/09514 dated 18 Aug. 1997, or a derivative thereof for the manufacture of a composition for inhibiting the growth of methane-producing bacteria and/or archaea in the gastrointestinal tract of monogastric animals, reducing the ability of the microbiome of the gastrointestinal tract to produce methane, reducing methane production by a monogastric animal, increasing feed efficiency in a monogastric animal, or improving the body weight and/or body composition of a monogastric animal.

In a further aspect, the invention provides Lacticaseibacillus rhamnosus strain HN001, AGAL deposit number NM97/09514 dated 18 Aug. 1997, or a derivative thereof for use in inhibiting the growth of methane-producing bacteria and/or archaea in the gastrointestinal tract of monogastric animals, reducing the ability of the microbiome of the gastrointestinal tract to produce methane, reducing methane production by a monogastric animal, increasing feed efficiency in a monogastric animal, or improving the body weight and/or body composition of a monogastric animal.

In a further aspect the invention provides a method for reducing methane emissions by a monogastric animal, wherein the method comprises administering to the animal an effective amount of Lacticaseibacillus rhamnosus strain HN001, AGAL deposit number NM97/09514 dated 18 Aug. 1997, or a derivative thereof.

In a further aspect, the invention provides use of Lacticaseibacillus rhamnosus strain HN001, AGAL deposit number NM97/09514 dated 18 Aug. 1997, or a derivative thereof for the manufacture of a composition for inhibiting the growth of methane-producing bacteria and/or archaea in the gastrointestinal tract of monogastric animals, reducing the ability of the microbiome of the gastrointestinal tract to produce methane, reducing methane emissions by a monogastric animal, increasing feed efficiency in a monogastric animal, or improving the body weight and/or body composition of a monogastric animal.

In a further aspect, the invention provides Lacticaseibacillus rhamnosus strain HN001, AGAL deposit number NM97/09514 dated 18 Aug. 1997, or a derivative thereof for use in inhibiting the growth of methane-producing bacteria and/or archaea in the gastrointestinal tract of monogastric animals, reducing the ability of the microbiome of the gastrointestinal tract to produce methane, reducing methane emissions by a monogastric animal, increasing feed efficiency in a monogastric animal, or improving the body weight and/or body composition of a monogastric animal.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

FIG. 1 shows (A) the relative abundances of reads assigned to Lactobacillus rhamnosus in caecal samples, and (B) the collective relative abundances of reads assigned to Lactobacillus rhamnosus and uncultured and unclassified Lactobacillus in in caecal samples.

Significant differences indicated by asterisk (permutation ANOVA p<0.01). Boxplots indicate median (middle line), first and third quartile (boundaries of box), 1.5 times the interquartile range (whiskers), and outliers (circles).

FIG. 2 shows principle component analysis (PCA) scores plot of the piglet caecal microbiome community composition at the genus level (PC1 vs PC2 and PC1 vs PC3 shown). Colours indicate groups; Control (medium grey), HN001™ Low (light grey), HN001™ High (dark grey). Permutation MANOVA p=0.001 indicates groups had significantly different compositions. Pairwise permutation MANOVAs showed the HN001™ Low and HN001™ High were not different to each other (p=0.283), whereas Controls differed from both HN001™ High (p=0.012) and HN001™ Low (p=0.002).

FIG. 3 shows differences in caecal abundance of different bacterial groups, between HN001™ (DR20) High, HN001™ (DR20) Low, and control treatments (FDR<0.05).

FIG. 4 shows differences in caecal abundance of Methanobrevibacter between HN001™ (DR20) High, HN001™ (DR20) Low, and control treatment.

FIG. 5 shows changes in abundance of genes related to methane metabolism between HN001™ (DR20) High, HN001™ (DR20) Low, and control treatments.

FIG. 6 shows KEGG pathways differentially expressed by GSEA (p<0.05) in at least one treatment and tissue. Black circles indicate overall significantly higher expression compared to controls and white circles indicate overall significantly lower expression compared to controls. Grey circles indicate pathway not differentially expressed (P>0.05). The size of the circle is proportional to the number of genes up or down regulated.

FIG. 7 shows heatmaps displaying the mean expression profiles and hierarchical clustering of genes within the KEGG (A) Tight junction, (B) Regulation of autophagy, (C) Basal transcription factors, and (D) RNA transport pathways. The ribbon across the right of the heatmap indicates the sample treatment group; Control (medium grey, top), HN001™ (DR20) Low (light grey, middle), and HN001™ (DR20) High (dark grey, bottom).

FIG. 8 shows the effect of feed supplementation with L. rhamnosus HN001™ on the growth of pigs.

FIG. 9 shows the effect of L. rhamnosus HN001™ on the most probable number (MPN) of methanogenic bacteria per gram of pig faeces in the caecum.

FIG. 10 shows the effect of L. rhamnosus HN001™ on the most probable number (MPN) of methanogenic bacteria per gram of pig faeces in the colorectum.

FIG. 11 shows the main volatile fatty acid concentrations (mM) measured in pig caecal samples collected after euthanasia. n=8 animals per group.

FIG. 12 shows the lactic acid concentration (mM) measured in pig caecal samples collected after euthanasia. n=8 animals per group.

FIG. 13 shows the main volatile fatty acid concentration (mM) measured in pig colorectal samples collected after euthanasia. n=8 animals per group.

FIG. 14 shows the lactic acid concentration (mM) measured in pig colorectal samples collected after euthanasia. n=8 animals per group.

FIG. 15 shows the succinic acid concentration (mM) measured in pig colorectal samples collected after euthanasia. n=8 animals per group.

### DETAILED DESCRIPTION OF THE INVENTION

The present invention is based on the finding that lactic acid bacteria strain Lacticaseibacillus rhamnosus HN001 (formerly classified as Lactobacillus rhamnosus HN001) and derivatives thereof inhibits the growth of methane-producing bacteria and/or archaea in the gastrointestinal tract of monogastric animals. Inhibiting the growth of methane-producing bacteria and/or archaea, and/or reducing the ability of the microbiome of the gastrointestinal tract to produce methane, can reduce methane production and increase volatile fatty acids (VFAs) in the gastro-intestinal tract, which can act as an increased energy source driving enhanced growth or increased productivity, such as meat production.

Accordingly, in a first aspect the invention provides a method for inhibiting the growth of methane-producing bacteria and/or archaea in the gastrointestinal tract of monogastric animals, or reducing the ability of the microbiome of the gastrointestinal tract to produce methane, wherein the method comprises administering to a monogastric animal an effective amount of Lacticaseibacillus rhamnosus strain HN001, AGAL deposit number NM97/09514 dated 18 Aug. 1997, or a derivative thereof.

In a second aspect, the invention provides a method for reducing methane production by a monogastric animal, wherein the method comprises administering to the animal an effective amount of Lacticaseibacillus rhamnosus strain HN001, AGAL deposit number NM97/09514 dated 18 Aug. 1997, or a derivative thereof.

In a third aspect the invention provides a method for increasing feed efficiency in a monogastric animal, wherein the method comprises administering to the animal an effective amount of Lacticaseibacillus rhamnosus strain HN001, AGAL deposit number NM97/09514 dated 18 Aug. 1997, or a derivative thereof.

The term “reducing methane production”, e.g., “reducing methane production by a monogastric animal by a monogastric animal”, refers to reducing methane production by any mechanism, and from any monogastric-animal-related source. For example, the term may refer to a reduction in methane produced within the gastrointestinal tract of the monogastric animal, or it may refer to a reduction in methane produced or emitted by the faeces or manure of a monogastric animal.

The term “administering” refers to the action of introducing an effective amount of Lacticaseibacillus rhamnosus strain HN001 into the gastro-intestinal tract of a monogastric animal. More particularly, this administration is an administration by oral route. This administration can in particular be carried out by supplementing the feed or drink intended for the animal with the strain; the supplemented feed or drink then being ingested by the animal.

The term “effective amount” refers to a quantity of Lacticaseibacillus rhamnosus strain HN001 sufficient to allow a desired effect, i.e., inhibition of the growth of methane-producing bacteria and/or archaea in the gastro-intestinal tract of the animal, a reduction in gastro-intestinal methane production or emission by the animal, or an increase in feed efficiency in the animal, in comparison with a reference. The desired effect (such as inhibition of growth of methane-producing bacteria and/or archaea and/or reduction of methane production or emission) can be measured in vitro or in vivo. For example, the desired effect can be measured in vitro using the methods described herein, for example, in the Examples below, or by in vivo oral administration to animals.

This effective amount can be administered to the monogastric animal in one or more doses.

It is anticipated that the reduction in methane production may be due to a variety of mechanisms. These may include, for example, killing methanogens (i.e. a bactericidal/archaeacidal effect), inhibiting the growth of methanogens (i.e. a bacteriostatic/archaeostatic effect), and/or inhibiting the ability of the gastrointestinal microbiota to produce methane. Inhibiting the ability of the gastrointestinal microbiota to produce methane may be via a variety of mechanisms, including, for example, physical and/or chemical changes to the gastrointestinal or caecal environment, changes to the microbiota, the inhibition of one or more methanogenic pathways, and/or cross-feeding (or disrupting cross-feeding) of intermediaries between members of the microbiome.

The term “feed efficiency” refers to the ability of an animal to turn feed nutrients into protein (such as muscle) and/or fat. Microbial fermentation in the gastro-intestinal tract produces volatile fatty acids (VFA) such as acetic acid, propionic acid and butyric acid. These fatty acids and their conjugate bases (acetate, propionate, butyrate) are absorbed directly from the GI tract and subsequently utilised by the host as substrate for metabolic energy production. Thus, when the utilisation of energy is improved, increases in muscle, and/or improvements in body composition, such as altered muscle/fat ratio in an animal, can also be achieved.

Feed efficiency can be calculated by dividing the total weight gain by an animal by the weight of dry matter consumed by that animal. Thus, an animal with a higher feed efficiency will gain more weight than an animal with a lower feed efficiency when given the same nutrient input. Feed efficiency can also be measured by differences in the growth of an animal by any of the following parameters: average daily weight gain, total weight gain, feed conversion, which includes both feed:gain and gain:feed, feed efficiency, mortality, and feed intake.

In one embodiment, the feed efficiency in a monogastric animal is increased to at least about 1.01× of the feed efficiency of an untreated animal, such as at least about 1.02×, 1.03×, 1.04×, 1.05×, 1.06×, 1.07×, 1.08×, 1.09×, 1.10×, 1.12×, 1.14×, 1.16×, 1.18×, such as at least about 1.20×. In some embodiments, L. rhamnosus HN001 or a derivative thereof promotes propionic acid production. Propionic acid has higher ATP production efficiency compared with other volatile fatty acids, and hence, feed efficiency is improved owing to the promotion of propionic acid production.

In some embodiments, L. rhamnosus HN001 or a derivative thereof shifts hydrogen metabolism from methanogenesis to short chain/volatile fatty acid (VFA) production, for example to propionic acid production. Propionate is predominantly used as a glucose precursor, and more propionate formation would likely result in a more efficient utilisation of feed energy. Maximizing the flow of metabolic hydrogen in the gastrointestinal tract away from methane and toward VFA (mainly propionate) would increase feed efficiency of animal farming and decrease its environmental impact.

It is anticipated that the methods of the present invention could be used to reduce or ameliorate the deterioration of body condition due to birthing or laying. It is anticipated that the methods disclosed herein will increase feed efficiency by the monogastric animal and therefore result in the monogastric animal having an improved body condition at the end of birthing or laying. As a result, the monogastric animal would require less feed intake to gain body condition. Alternatively or additionally, the methods and feed compositions disclosed herein are useful for improving body condition of an animal prior to lactation. For example, the methods and compositions disclosed herein could improve the body composition of the mother and/or the foetus or neonate. For example, the methods and compositions disclosed herein could improve body composition and/or weight of the neonate at birth.

It is also anticipated that the methods of the present invention could be similarly useful for reducing or ameliorating the deterioration of body condition in other times of stress, such as drought or insufficient feed intake.

As used herein, the term “gastrointestinal tract” refers to the part of the monogastric digestive system that begins in the stomach and ends in the rectum, including the small intestine. Therefore, the mouth and oesophagus are not considered part of the gastrointestinal tract for the purposes of this application.

In some embodiments, the growth of methane-producing bacteria and/or archaea is inhibited in faeces of the animal. In some embodiments, the growth of methane-producing bacteria and/or archaea is inhibited in the distal intestine of the animal. In some embodiments the growth of methane-producing bacteria and/or archaea is inhibited in the colon of the animal. In some embodiments, the growth of methane-producing bacteria and/or archaea is inhibited in the rectum of the animal. In some embodiments, the growth of methane-producing bacteria and/or archaea is inhibited in the small intestine of the animal. In some embodiments, the growth of methane-producing bacteria and/or archaea is inhibited in the hindgut of the animal. In some embodiments, the growth of methane-producing bacteria and/or archaea is inhibited in the caecum of the animal.

It is also anticipated that the methods of the present invention could be useful for improving gut comfort, or preventing, reducing or ameliorating the symptoms caused by gases produced by methanogens in the gastrointestinal tract of the animal, for example excessive flatulence, abdominal distension (bloating) and abdominal pain.

### Monogastric Animals

Monogastric animals are a group of animals having a simple single-chambered stomach, in comparison with ruminant animals, which have a stomach comprising multiple compartments including a foregut or rumen. The monogastric animals group includes carnivores, omnivores, and herbivores, such as humans, cats, dogs, pigs, horses, donkeys, rabbits, and poultry.

Monogastric animals include several species of domesticated livestock. In one embodiment, the monogastric animal is a human, pig, horse, donkey, rabbit, or poultry. In a preferred embodiment, the monogastric animal is a pig. In one embodiment, the monogastric animal is a chicken, duck, goose or turkey. In one embodiment, the monogastric animal is a companion animal, such as a cat or a dog.

In one embodiment, the monogastric animal is a pre-weaning animal, such as a piglet or a foal. In some embodiments, the L. rhamnosus HN001 or derivative thereof is administered to the monogastric animal prior to weaning. In some embodiments, the L. rhamnosus HN001 or derivative thereof is administered to the monogastric animal after weaning. In some embodiments, the L. rhamnosus HN001 or derivative thereof is administered to the monogastric animal both prior to weaning and after weaning.

For example, the L. rhamnosus HN001 or derivative thereof is administered to the animal on or about day 0 of birth, for example around day 0, day 1 or day 2 of birth. Administration may then occur at least one per day, for example multiple times per day, sufficient to obtain persistency of effect. For example, administration may continue for 2 days, 3 days, 4 days, 5 days, 6 days, 1 week, 2 weeks, 3 weeks, one month, 6 weeks, 2 months, 10 weeks or three months from birth.

### Lacticaseibacillus rhamnosus HN001

As described in the applicant's PCT International application PCT/NZ98/00122 (published as WO 99/10476 and incorporated herein in its entirety), a freeze-dried culture of Lacticaseibacillus rhamnosus HN001 (formerly classified as Lactobacillus rhamnosus HN001) was deposited at the Australian Government Analytical Laboratories (AGAL), The New South Wales Regional Laboratory, 1 Suakin Street, Pymble, NSW 2073, Australia, on 18 Aug. 1997 and was accorded deposit number NM97/09514. This Budapest Treaty-recognised depository is now no longer referred to as AGAL, but rather is referred to as the National Measurement Institute of Australia (NMIA). The genome sequence of L. rhamnosus HN001 is available at Genbank under accession number: NZ_ABWJ00000000. The terms Lacticaseibacillus rhamnosus HN001, Lactobacillus rhamnosus HN001, L. rhamnosus HN001, DR20™ and HN001™ are used interchangeably herein. DR20™ and HN001™ are trade marks of Fonterra™ Limited.

### Morphological Properties

The morphological properties of L. rhamnosus HN001 are described below.

Short to medium rods with square ends in chains, generally 0.7×1.1×2.0-4.0 μm, when grown in MRS broth.

Gram positive, non-mobile, non-spore forming, catalase negative facultative anaerobic rods with optimum growth temperature of 37±1° C. and optimum pH of 6.0-6.5. These are facultatively heterofermentative bacteria and no gas is produced from glucose.

### Fermentative Properties

An API 50 CH sugar fermentation kit was used to determine the carbohydrate fermentation pattern of L. rhamnosus HN001, yielding a score of 5757177 (based on scores of 22 prominent sugars—see PCT/NZ98/00122).

### Further Characterisation

L. rhamnosus strain HN001 may be further characterised by the functional attributes disclosed in PCT/NZ98/00122, including its ability to adhere to human intestinal epithelial cells, and by the improvements in phagocyte function, in antibody responses, in natural killer cell activity, and in lymphocyte proliferation elicited by dietary intake or in in vitro model systems. It will be appreciated that there are a wide variety of methods known and available to the skilled artisan that can be used to confirm the identity of L. rhamnosus HN001, wherein exemplary methods include DNA fingerprinting, genomic analysis, sequencing, and related genomic and proteomic techniques.

### L. rhamnosus HN001 and Derivatives Thereof

As described herein, certain embodiments of the present invention utilise live L. rhamnosus HN001. In other embodiments, an L. rhamnosus HN001 derivative is utilised.

As used herein, the term “derivative” and grammatical equivalents thereof when used with reference to bacteria (including use with reference to a specific strain of bacteria such as L. rhamnosus HN001) contemplates mutants and homologues of or derived from the bacteria, killed or attenuated bacteria such as but not limited to heat-killed, lysed, fractionated, pressure-killed, irradiated, and UV- or light-treated bacteria, and material derived from the bacteria including but not limited to bacterial cell wall compositions, bacterial cell lysates, lyophilised bacteria, anti-methanogen factors from the bacteria, bacterial metabolites, bacterial cell suspensions, bacterial culture supernatant, and the like, wherein the derivative retains anti-methanogen activity. Transgenic microorganisms engineered to express one or more anti-methanogen factors are also contemplated. Methods to produce such derivatives, such as but not limited to one or more mutants of L. rhamnosus HN001 or one or more anti-methanogen factors, and particularly derivatives suitable for administration to a monogastric animal (for example, in a composition) are well known in the art.

It will be appreciated that methods suitable for identifying L. rhamnosus HN001, such as those described above, are similarly suitable for identifying derivatives of L. rhamnosus HN001, including for example mutants or homologues of L. rhamnosus HN001, or for example bacterial metabolites from L. rhamnosus HN001.

The term “anti-methanogen factor” refers to a bacterial molecule responsible for mediating anti-methanogen activity, including but not limited to bacterial DNA motifs, proteins, bacteriocins, bacteriocin-like molecules, anti-microbial peptides, antibiotics, antimicrobials, small molecules, polysaccharides, or cell wall components such as lipoteichoic acids and peptidoglycan, or a mixture of any two or more thereof. While, as noted above, these molecules have not been clearly identified, and without wishing to be bound by any theory, their presence can be inferred by the presence of anti-methanogen activity.

The term “anti-methanogen activity” refers to the ability of certain microorganisms to inhibit the growth of methanogenic bacteria and/or archaea, and/or to reduce the production of methane by methanogenic bacteria and/or archaea. This ability may be limited to inhibiting the growth and/or ability to produce methane of certain groups of methanogenic bacteria and/or archaea such as, for example, inhibiting the growth of hydrogenotrophic methanogens, inhibiting the ability of hydrogenotrophic methanogens to produce methane, inhibiting the growth of methylotrophic methanogens, inhibiting the ability of methylotrophic methanogens to produce methane, inhibiting the growth of certain species of methanogens, or inhibiting the ability of certain species of methanogens to produce methane.

Reference to retaining anti-methanogen activity is intended to mean that a derivative of a microorganism, such as a mutant or homologue of a microorganism or an attenuated or killed microorganism, or a cell culture supernatant, still has useful anti-methanogen activity, or that a composition comprising a microorganism or a derivative thereof still has useful anti-methanogen activity. While the bacterial molecules responsible for mediating anti-methanogen activity have not been clearly identified, molecules that have been proposed as possible candidates include bacterial DNA motifs, proteins, bacteriocins, antibiotics, surface proteins, small organic acids, polysaccharides, and cell wall components such as lipoteichoic acids and peptidoglycan. It has been postulated that these interact with components of the methanogenic bacteria and/or archaea to give a growth-inhibitory effect. Preferably, the retained activity is at least about 35, 40, 45, 50, 55, 60, 65, 70, 75, 80, 85, 90, 95, 99 or 100% of the activity of an untreated (i.e., live or non-attenuated) control, and useful ranges may be selected between any of these values (for example, from about 35 to about 100%, from about 50 to about 100%, from about 60 to about 100%, from about 70 to about 100%, from about 80 to about 100%, and from about 90 to about 100%).

Using conventional solid substrate and liquid fermentation technologies well known in the art, L. rhamnosus HN001 can be grown in sufficient amounts to allow use as contemplated herein. For example, L. rhamnosus HN001 can be produced in bulk for formulation using nutrient film or submerged culture growing techniques, for example under conditions as described in WO99/10476. Briefly, growth is carried out under aerobic conditions at any temperature satisfactory for growth of the organism. For example, for L. rhamnosus HN001 a temperature range of from 30 to 40° C., preferably 37° C., is preferred. The pH of the growth medium is slightly acidic, preferably about 6.0 to 6.5. Incubation time is sufficient for the isolate to reach a stationary growth phase.

L. rhamnosus HN001 cells may be harvested by methods well known in the art, for example, by conventional filtering or sedimentary methodologies (e.g. centrifugation) or harvested dry using a cyclone system. L. rhamnosus HN001 cells can be used immediately or stored, preferably freeze-dried or chilled at −20° to 6° C., preferably −4° C., for as long as required using standard techniques.

### Supernatants

Further embodiments of the present invention utilise supernatant(s) from a cell culture comprising L. rhamnosus HN001 or a derivative thereof. These embodiments include processes for preparing an L. rhamnosus HN001 supernatant, said process comprising culturing cells of L. rhamnosus HN001, and separating the supernatant from the cultured cells, thereby obtaining the supernatant. This process also enables further isolation of bacterial molecules responsible for mediating anti-methanogen activity that are obtainable from the supernatant.

As would be understood by the skilled addressee, a supernatant useful in the present invention encompasses both the supernatant from such cultures, and/or concentrates of such supernatant and/or fractions of such supernatant.

The term “supernatant” in the present context refers to a medium from a bacterial culture from which the bacteria have subsequently been removed, e.g. by centrifugation or filtration.

A supernatant useful in the present invention can readily be obtained by a simple process for preparing an L. rhamnosus HN001 supernatant, said process comprising
a) culturing cells of L. rhamnosus HN001, and
b) optionally releasing of active compounds and/or extracellular components of the cells by various cellular treatments such as, but not limited to, acidic or alkaline modifications, sonication, detergents e.g. Sodium dodecyl sulfate (SDS) and/or Triton X, muralytic enzymes e.g. mutalolysin and/or lysozyme, salt and/or alcohol;
c) separating the supernatant from the cultured cells,
   thereby obtaining said supernatant.

In a preferred embodiment of this process, the supernatant composition is further subjected to a drying step to obtain a dried culture product.

The drying step may conveniently be freeze drying or spray drying, but any drying process which is suitable for drying of anti-methanogen factors such as bacteriocins, also including vacuum drying and air drying, are contemplated.

Although the content of the supernatant produced by L. rhamnosus HN001 is not yet characterised in detail, it is known that certain Lactobacillus may produce bacteriocins that are small heat-stable proteins and therefore, without wishing to be bound by theory, it is expected that even drying methods, including spray drying, which result in moderate heating of the culture eluate product, will result in active compositions.

### Lysate

A fluid containing the contents of lysed cells is called a lysate. A lysate contains active components of the bacterial cells and may be either crude, thus containing all cellular components, or partially and/or completely separated in separate fractions, such as extracellular components, intracellular components, proteins etc.

Methods for producing bacterial cell lysates are well known in the art. Such methods can include, but are not limited to, mechanical lysis, such as mechanical shearing, grinding, milling, or sonication, enzymatic lysis, such as by enzymes that degrade the bacterial cell wall, chemical lysis, such as using detergents, denaturants, pressure alterations, and/or osmotic shock, and combinations of the above.

Further embodiments of the present invention thus utilise a lysate of L. rhamnosus HN001 or a derivative thereof.

### Cell Suspension

The present invention may also in some embodiments utilise a cell suspension comprising L. rhamnosus HN001 or a derivative thereof.

In the present context, the term “cell suspension” relates to a number of L. rhamnosus HN001 or a derivative thereof dispersed or in suspension in a liquid e.g. a liquid nutrient medium, culture medium or saline solution.

The cells may be presented in the form of a cell suspension in a solution that is suitable for dispersion. The cell suspension can e.g. be dispersed via spraying, dipping, or any other application process.

The cells may be viable, but the suspension may also comprise inactivated or killed cells or a lysate hereof. In one embodiment, the suspension of the present invention comprises viable cells. In another embodiment, the suspension of the present invention comprises inactivated, killed or lysed cells.

### Bacteriocins

Bacteriocins are antimicrobial compounds produced by bacteria to inhibit other bacterial strains and species.

Lactic acid bacteria (LAB) are well known to produce bacteriocins and these compounds are of global interest to the food industry because they inhibit the growth of many spoilage and pathogenic bacteria, thus extending shelf life and safety of foods. Bacteriocins are typically considered to be narrow spectrum antibiotics. Moreover, bacteriocins of especially LAB display very low human toxicity and have been consumed in fermented food for millennia.

As is illustrated in the Examples disclosed herein, it has been found that L. rhamnosus HN001, or compositions comprising L. rhamnosus HN001, and the culture supernatant of L. rhamnosus HN001 are useful as an antimicrobial compound, in particular for inhibiting the growth of methane-producing bacteria, and/or inhibiting the ability of methanogens to produce methane.

In the present context, the term antimicrobial compound utilises a compound that kills microorganisms, impair their survival or inhibits their growth.

Antimicrobial compounds can be grouped according to the microorganisms they act primarily against. For example, antibacterials are used against bacteria and antifungals are used against fungi. They can also be classified according to their function. Compounds that kill microbes are called microbicidal, while those that merely inhibit their growth are called microbiostatic.

In one embodiment, the present invention relates to an antimicrobial compound, which is microbicidal. In another embodiment, the present invention relates to an antimicrobial compound, which is microbiostatic. In another embodiment, the present invention relates to an antimicrobial compound, which is antibacterial.

### Monogastric Feed or Carrier Compositions

A monogastric composition useful herein may be formulated as a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet or bolus. Appropriate formulations may be prepared by an art skilled worker with regard to that skill and the teaching of this specification.

The composition can be administered as a top dressing on, or mixed into, a standard feed material such as a daily ration. In addition, the strain can be administered in a partial or total mixed ration (TMR), pelleted feedstuff, mixed in with liquid feed or drink, mixed in a protein premix, or delivered via a vitamin and mineral premix.

In one embodiment, compositions useful herein include any edible feed product which is able to carry bacteria or a bacterial derivative. As used in this application, the term “feed(s)” or “animal feed(s)” refers to material(s) that are consumed by animals and contribute energy and/or nutrients to an animal's diet. Animal feeds typically include a number of different components that may be present in forms such as concentrate(s), premix(es), co-product(s), or pellets. Examples of feeds and feed components include Partial or Total Mixed Ration (TMR), corn, soybean, forage, grain, distiller's grain, sprouted grain, legumes, vitamins, amino acids, minerals, fibre, fodder, grass, hay, silage, kernel, leaves, meal, solubles, slurries, supplements, mash feed, meal, fruit pulp, vegetable pulp, fruit or vegetable pomace, citrus meal, wheat shorts, corn cob meal, and molasses. Other compositions useful as a carrier include milk, milk powder, milk replacement, milk fortifier, whey, whey powder, sucrose, maltodextrin, and rice hulls.

In certain embodiments, the feed composition is formed through a process of growing L. rhamnosus HN001 using a milk-based carrier, such as thermalized milk, or a non-milk-based carrier, to create a fermented yoghurt-style composition. Methods to create such fermented yoghurt-style compositions are well known in the art, and may include, for example, using a warm water bath or other heating means to incubate the milk at a suitable temperature until a sufficient cell density is reached, such as over 12 hours. In one embodiment, the temperature is 25-30° C. Optionally, the milk may include other additives to promote bacterial growth, such as yeast extract. In certain embodiments, this method takes place on-site, such as on the farm where the probiotic feed supplementation is to take place. The fermented yoghurt-style composition may be administered by oral application, such as by drenching. In some embodiments, the fermented yoghurt-style composition is administered at a dose of 1-100 ml per day, such as 2-50, 5-30, or 10-20 ml per day.

In one embodiment, compositions useful herein include any non-feed carrier consumed by the animal to which bacteria or a bacterial derivative is added, such as vermiculite, zeolites or crushed limestone and the like.

In one embodiment, compositions useful herein include pet food compositions for companion animals such as cats and dogs. In certain embodiments, the L. rhamnosus HN001 is included in the pet food in an amount of about 10⁴ cfu (colony forming units)/g of pet food to about 10¹⁴ cfu/g of pet food. In certain embodiments, the composition further comprises at least one protein source. In certain embodiments, the composition further comprises at least one source of fat. In certain embodiments, the composition further comprises at least one carbohydrate source. In certain embodiments, the pet food is a dog food. In certain embodiments, the pet food is a cat food.

The terms “pet food”, or “pet food composition” as used herein means nutritional compositions intended for ingestion by a pet. In one embodiment a nutritional composition may refer to a dietary supplement intended for ingestion by a pet. A dietary supplement is intended to refer to a composition that provides nutrients that may otherwise not be consumed in sufficient quantities by the pet. In one embodiment a nutritional composition may refer to a pet treat intended for ingestion by a pet. The term “pet treat” as used herein refers to a food for consumption by a pet that is intended as an occasional reward or indulgence and not as the sole source of a pet's nutrition.

In one embodiment, compositions useful herein include food compositions for omnivores such as chickens, pigs, humans, and dogs. Such food compositions are well known in the art.

In certain embodiments, the composition of the invention comprises live L. rhamnosus HN001. Methods to produce such compositions are well-known in the art.

In some embodiments, the composition of the invention comprises one or more L. rhamnosus HN001 derivatives. Again, methods to produce such compositions are well known in the art and may utilise standard microbiological and pharmaceutical practices. In some embodiments, the composition comprises a dried culture product, such as a supernatant or cell lysate as described herein.

It will be appreciated that a broad range of additives or carriers may be included in such compositions, for example to improve or preserve bacterial viability or to increase anti-methanogen activity of L. rhamnosus HN001 or of one or more L. rhamnosus HN001 derivatives. For example, additives such as surfactants, wetters, humectants, stickers, dispersal agents, stabilisers, penetrants, and so-called stressing additives to improve bacterial cell vigour, growth, replication and survivability (such as potassium chloride, glycerol, sodium chloride and glucose), as well as cryoprotectants such as maltodextrin, may be included. Additives may also include compositions which assist in maintaining microorganism viability in long term storage, for example unrefined corn oil, or “invert” emulsions containing a mixture of oils and waxes on the outside and water, sodium alginate and bacteria on the inside.

In some embodiments, the L. rhamnosus HN001 or derivative thereof are encapsulated. Methods to produce such encapsulated bacteria are well known in the art. In some embodiments, the L. rhamnosus HN001 or derivative thereof are encapsulated in liposomes, microbubbles, microparticles or microcapsules and the like. Such encapsulants can include natural, semisynthetic, or synthetic polymers, waxes, lipids, fats, fatty alcohols, fatty acids, and/or plasticisers, for example alginates, gums, κ-Carrageenan, chitosan, starch, sugars, gelatine, and so on.

In certain embodiments, the L. rhamnosus HN001 is in a reproductively viable form and amount.

The composition may comprise a carbohydrate source, such as a disaccharide including, for example, sucrose, fructose, glucose, or dextrose. Preferably the carbohydrate source is one able to be aerobically or anaerobically utilised by L. rhamnosus HN001.

In such embodiments, the composition preferably is capable of supporting reproductive viability of the L. rhamnosus HN001 for a period greater than about two weeks, preferably greater than about one month, about two months, about three months, about four months, about five months, more preferably greater than about six months, most preferably at least about 2 years to about 3 years or more.

In certain embodiments, an oral composition is formulated to allow the administration of an effective amount of L. rhamnosus HN001 to establish a population in the gastrointestinal tract of the animal when ingested. The established population may be a transient or permanent population.

While various routes and methods of administration are contemplated, oral administration of L. rhamnosus HN001, such as in a composition suitable for oral administration, is currently preferred. It will of course be appreciated that other routes and methods of administration may be utilised or preferred in certain circumstances.

The term “oral administration” includes oral, buccal, enteral, and intra-gastric administration.

In theory one colony forming unit (cfu) should be sufficient to establish a population of L. rhamnosus HN001 in an animal, but in actual situations a minimum number of units are required to do so. Therefore, for therapeutic mechanisms that are reliant on a viable, living population of probiotic bacteria, the number of units administered to a subject will affect efficacy.

In one embodiment, a composition formulated for administration will be sufficient to provide at least about 6×10⁹ cfu L. rhamnosus HN001 per day. In another embodiment, a composition formulated for administration will be sufficient to provide at least about 10¹⁰ cfu L. rhamnosus HN001 per day.

Methods to determine the presence of a population of gut flora, such as L. rhamnosus HN001, in the gastrointestinal tract of a subject are well known in the art, and examples of such methods are presented herein. In certain embodiments, presence of a population of L. rhamnosus HN001 can be determined directly, for example by analysing one or more samples obtained from an animal and determining the presence or amount of L. rhamnosus HN001 in said sample. In other embodiments, presence of a population of L. rhamnosus HN001 can be determined indirectly, for example by observing a reduction in methane emissions or methane production, a reduction in hydrogen production, or a decrease in the number of other gut flora in a sample obtained from an animal. Combinations of such methods are also envisaged.

The efficacy of a composition useful according to the invention can be evaluated both in vitro and in vivo. See, for example, the examples below. Briefly, the composition can be tested for its ability to inhibit the growth of methanogenic bacteria and/or archaea, or its ability to reduce the production of methane by methanogenic bacteria and/or archaea. For in vivo studies, the composition can be fed to or injected into a monogastric animal and its effects on methanogenic bacteria and/or archaea, and its effect on methane production or emission are then assessed. Based on the results, an appropriate dosage range and administration route can be determined.

Methods of calculating appropriate dose may depend on the nature of the active agent in the composition. For example, when the composition comprises live L. rhamnosus HN001, the dose may be calculated with reference to the number of live bacteria present. For example, as described herein the examples the dose may be established by reference to the number of colony forming units (cfu) to be administered per day, or by reference to the number of cfu per kilogram dry feed weight.

By way of general example, the administration of from about 1×10⁶ cfu to about 1×10¹² cfu of L. rhamnosus HN001 per kg dry feed weight per day, preferably about 1×10⁶ cfu to about 1×10¹¹ cfu/kg/day, about 1×10⁶ cfu to about 1×10¹⁰ cfu/kg/day, about 1×10⁶ cfu to about 1×10⁹ cfu/kg/day, about 1×10⁶ cfu to about 1×10⁸ cfu/kg/day, about 1×10⁶ cfu to about 5×10⁷ cfu/kg/day, or about about 1×10⁶ cfu to about 1×10⁷ cfu/kg/day, is contemplated. Preferably, the administration of from about 5×10⁶ cfu to about 5×10⁸ cfu per kg dry feed weight of L. rhamnosus HN001 per day, preferably about 5×10⁶ cfu to about 4×10⁸ cfu/kg/day, about 5×10⁶ cfu to about 3×10⁸ cfu/kg/day, about 5×10⁶ cfu to about 2×10⁸ cfu/kg/day, about 5×10⁶ cfu to about 1×10⁸ cfu/kg/day, about 5×10⁶ cfu to about 9×10⁷ cfu/kg/day, about 5×10⁶ cfu to about 8×10⁷ cfu/kg/day, about 5×10⁶ cfu to about 7×10⁷ cfu/kg/day, about 5×10⁶ cfu to about 6×10⁷ cfu/kg/day, about 5×10⁶ cfu to about 5×10⁷ cfu/kg/day, about 5×10⁶ cfu to about 4×10⁷ cfu/kg/day, about 5×10⁶ cfu to about 3×10⁷ cfu/kg/day, about 5×10⁶ cfu to about 2×10⁷ cfu/kg/day, or about 5×10⁶ cfu to about 1×10⁷ cfu/kg/day, is contemplated.

In certain embodiments, periodic dose need not vary with body weight, dry feed weight or other characteristics of the subject. In such examples, the administration of from about 1×10⁶ cfu to about 1×10¹³ cfu of L. rhamnosus HN001 per day, preferably about 1×10⁶ cfu to about 1×10¹² cfu/day, about 1×10⁶ cfu to about 1×10¹¹ cfu/day, about 1×10⁶ cfu to about 1×10¹⁰ cfu/day, about 1×10⁶ cfu to about 1×10⁹ cfu/day, about 1×10⁶ cfu to about 1×10⁸ cfu/day, about 1×10⁶ cfu to about 5×10⁷ cfu/day, or about about 1×10⁶ cfu to about 1×10⁷ cfu/day, is contemplated.

In certain embodiments, the administration of from about 5×10⁷ cfu to about 5×10¹⁰ cfu per kg body weight of L. rhamnosus HN001 per day, preferably about 5×10⁷ cfu to about 4×10¹⁰ cfu/day, about 5×10⁷ cfu to about 3×10¹⁰ cfu/day, about 5×10⁷ cfu to about 2×10¹⁰ cfu/day, about 5×10⁷ cfu to about 1×10¹⁰ cfu/day, about 5×10⁷ cfu to about 9×10⁹ cfu/day, about 5×10⁷ cfu to about 8×10⁹ cfu/day, about 5×10⁷ cfu to about 7×10⁹ cfu/day, about 5×10⁷ cfu to about 6×10⁹ cfu/day, about 5×10⁷ cfu to about 5×10⁹ cfu/day, about 5×10⁷ cfu to about 4×10⁹ cfu/day, about 5×10⁷ cfu to about 3×10⁹ cfu/day, about 5×10⁷ cfu to about 2×10⁹ cfu/day, or about 5×10⁷ cfu to about 1×10⁹ cfu/day, is contemplated. Preferably, a dose of between 1×10⁸ and 1×10⁹ cfu/kg body weight per day is administered.

It will be appreciated that, in certain embodiments, the dose need not be administered daily. For example, the composition may be formulated to be administered every two days, twice weekly, weekly, fortnightly, or monthly. Alternatively, in certain embodiments, the composition may be formulated to be administered with every feed, or with every mouthful.

In one embodiment, L. rhamnosus HN001 may be dosed at between about 1×10³ to about 1×10⁹ cfu/g of pet food and/or pet food admixture.

Suitably, L. rhamnosus HN001 may be dosed at between about 1×10⁴ to about 1×10⁸ cfu/g of pet food and/or pet food admixture.

Suitably, L. rhamnosus HN001 may be dosed at between about 7.5×10⁴ to about 1×10⁷ cfu/g of pet food and/or pet food admixture.

Preferably, the L. rhamnosus HN001 may be dosed at about 1×10⁶ cfu/g of pet food and/or pet food admixture.

In embodiments where the pet food is a pet treat the number of cfu/g dosed may be between about 2 times to about 20 times, suitably between about 4 times to about 15 times the number of cfu/g dosed in a pet food and/or pet food admixture. Preferably the number of cfu/g dosed may be about 10 times the number of cfu/g dosed in a pet food and/or pet food admixture.

It will be appreciated that the composition is preferably formulated so as to allow the administration of an efficacious dose of L. rhamnosus HN001 or one or more derivatives thereof. The dose of the composition administered, the period of administration, and the general administration regime may differ between animals depending on such variables as mode of administration chosen, and the age, sex, body weight, and species of an animal. Furthermore, as described above the appropriate dose may depend on the nature of the active agent in the composition and the manner of formulation.

In some embodiments, the dose of the composition does not vary over time. In other embodiments, the dose of the composition may vary over time. For example, in some embodiments, an initial dosing regimen may be followed by a maintenance dosing regimen. It will be appreciated that a higher dose may be required to establish a population of L. rhamnosus HN001 in the animal, and a lower dose may be sufficient to maintain said population. Accordingly, in some embodiments, the initial dosing regimen comprises administering a higher dose and/or a more frequent dose than the maintenance dosing regimen. Preferably, the initial dosing regimen is efficacious to establish a population of L. rhamnosus HN001 in the animal, and preferably the maintenance dosing regimen is efficacious to maintain a population of L. rhamnosus HN001 in the animal. In some embodiments, the maintenance dosing regimen comprises administering a dose every day, every second day, twice weekly, weekly, fortnightly, or monthly.

In some embodiments, the effect of the methods described herein persist after the administration of the L. rhamnosus HN001. Without wishing to be bound by theory, it is anticipated that administration of L. rhamnosus HN001 as described herein may result in long-lasting or even permanent changes in the gastrointestinal tract of the monogastric animal. In some embodiments, the effect persists for 2 days after the last administration of L. rhamnosus HN001, such as 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus HN001. In a preferred embodiment, the effect persists for the life of the animal.

In examples where the composition comprises one or more L. rhamnosus HN001 derivatives, the dose may be calculated by reference to the amount or concentration of L. rhamnosus HN001 derivative to be administered per day. For example, when the bacteria are inactivated, the quantities described previously are calculated before inactivation. For a composition comprising L. rhamnosus HN001 culture supernatant, the dose may be calculated by reference to the concentration of L. rhamnosus HN001 culture supernatant present in the composition. The concentration of L. rhamnosus HN001 culture supernatant present in the composition may be calculated, for example, on the basis of the cfu of the culture. For example, a dosage of culture supernatant equivalent to 1×10⁹ cfu/day can be calculated from the total yield of the culture and the total volume of the culture supernatant.

It will be appreciated that preferred compositions are formulated to provide an efficacious dose in a convenient form and amount. In certain embodiments, such as but not limited to those where periodic dose need not vary with body weight or other characteristics of the animal, the composition may be formulated for unit dosage. It should be appreciated that administration may include a single daily dose or administration of a number of discrete divided doses as may be appropriate. For example, an efficacious dose of L. rhamnosus HN001 may be formulated into a feed for oral administration.

However, by way of general example, the inventors contemplate administration of from about 1 mg to about 1000 mg of a composition useful herein per day, preferably about 50 to about 500 mg per day, alternatively about 150 to about 410 mg/day or about 110 to about 310 mg/day. In one embodiment, the inventors contemplate administration of from about 0.05 mg to about 250 mg per kg body weight of a composition useful herein. For example, administration to a human may comprise a single dose of 6×10⁹ CFU per day to adults or children as a 500 mg capsule.

In one embodiment a composition useful herein comprises, consists essentially of, or consists of at least about 0.1, 0.2, 0.5, 1, 5, 10, 15, 20, 25, 30, 35, 40, 45, 50, 55, 60, 65, 75, 80, 85, 90, 95, 99, 99.5, 99.8 or 99.9% by weight of L. rhamnosus HN001 or a derivative thereof and useful ranges may be selected between any of these foregoing values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%, from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%, from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90%, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99%, from about 30 to about 99%, from about 35 to about 99%, from about 40 to about 99%, and from about 45 to about 99%).

In one embodiment a composition useful herein comprises, consists essentially of, or consists of at least about 0.001, 0.01, 0.05, 0.1, 0.15, 0.2, 0.3, 0.4, 0.5, 1, 2, 3, 4, 5, 6, 7, 8, 9, 10, 11, 12, 13, 14, 15, 16, 17, 18 or 19 grams of L. rhamnosus HN001 or a derivative thereof and useful ranges may be selected between any of these foregoing values (for example, from about 0.01 to about 1 grams, about 0.01 to about 10 grams, about 0.01 to about 19 grams, from about 0.1 to about 1 grams, about 0.1 to about 10 grams, about 0.1 to about 19 grams, from about 1 to about 5 grams, about 1 to about 10 grams, about 1 to about 19 grams, about 5 to about 10 grams, and about 5 to about 19 grams).

In certain embodiments, a composition useful herein comprises, consists essentially of, or consists of at least about 10⁴, 10⁵, 10⁶, 10⁷, 10⁸, 10⁹, 10¹⁰, 10¹¹, 10¹², or 10¹³ colony forming units (cfu) of L. rhamnosus HN001 per kg dry weight of the composition, and useful ranges may be selected between any of these foregoing values (for example, from about 10⁵ to about 10¹³ cfu, from about 10⁶ to about 10¹² cfu, from about 10⁷ to about 10¹² cfu, from about 10⁸ to about 10¹¹ cfu, from about 10⁸ to about 10¹⁰ cfu, and from about 10⁸ to about 10⁹ cfu).

It will be apparent that the concentration of L. rhamnosus HN001 or one or more derivatives thereof in a composition formulated for administration may be less than that in a composition formulated for, for example, distribution or storage, and that the concentration of a composition formulated for storage and subsequent formulation into a composition suitable for administration must be adequate to allow said composition for administration to also be sufficiently concentrated so as to be able to be administered at an efficacious dose.

The compositions useful herein may be used alone or in combination with one or more other therapeutic agents. The therapeutic agent may be a food, drink, food additive, drink additive, food component, drink component, dietary supplement, vitamin or mineral premix, oil, oil blend, oil rich feed supplement, nutritional product, medical food, nutraceutical, medicament or pharmaceutical. The therapeutic agent may be a probiotic agent or a probiotic factor, and is preferably effective to inhibit the growth of methanogenic bacteria and/or archaea, or to reduce methane emissions, for example methane production by methanogenic bacteria and/or archaea. In some embodiments, the oil, oil blend, or oil rich feed supplement is palm kernel expeller (PKE) and/or PROLIQ.

When used in combination with another therapeutic agent, the administration of a composition useful herein and the other therapeutic agent may be simultaneous or sequential. Simultaneous administration includes the administration of a single dosage form that comprises all components or the administration of separate dosage forms at substantially the same time. Sequential administration includes administration according to different schedules, preferably so that there is an overlap in the periods during which the composition useful herein and other therapeutic agent are provided. Examples of other therapeutic agents include at least one additional microorganism of a different species or strain, a vaccine that inhibits methanogens or methanogenesis, and/or a natural or chemically-synthesised inhibitor of methanogenesis and/or methanogen inhibitor, such as bromoform.

Suitable agents with which the compositions useful herein can be separately, simultaneously or sequentially administered include one or more prebiotic agents, one or more probiotic agents, one or more postbiotic agents, one or more phospholipids, one or more gangliosides, other suitable agents known in the art, and combinations thereof.

Typically, the term prebiotic refers to a material that stimulates the growth and/or activity of bacteria in the animals' digestive system that have biologic activity. Prebiotics may be selectively fermented ingredients that allow specific changes, both in the composition and/or activity of the gastrointestinal microflora, which confer health benefits upon the host. Probiotics generally refer to microorganisms that contribute to intestinal microbial balance which in turn play a role in maintaining health, or providing other biologic activity. Many species of lactic acid bacteria (LAB) such as, Lactobacillus and Bifidobacterium are generally considered as probiotics, but some species of Bacillus, and some yeasts have also been found as suitable candidates. Postbiotics refer to non-viable bacterial products or metabolic byproducts from microorganisms such as probiotics, that have biologic activity in the host.

Useful prebiotics include galactooligosaccharides (GOS), short chain GOS, long chain GOS, fructooligosaccharides (FOS), short chain FOS, long chain FOS, inulin, galactans, fructans, lactulose, and any mixture of any two or more thereof. Some prebiotics are reviewed by Boehm G and Moro G (Structural and Functional Aspects of Prebiotics Used in Infant Nutrition, J. Nutr. (2008) 138(9):1818S-1828S), incorporated herein by reference. Other useful agents may include dietary fibre such as a fully or partially insoluble or indigestible dietary fibre.

Accordingly, in one embodiment L. rhamnosus HN001 or derivative thereof may be administered separately, simultaneously or sequentially with one or more agents selected from one or more probiotics, one or more prebiotics, one or more sources of dietary fibre, one or more galactooligosaccharides, one or more short chain galactooligosaccharides, one or more long chain galactooligosaccharides, one or more fructooligosaccharides, one or more short chain galactooligosaccharides, one or more long chain galactooligosaccharides, inulin, one or more galactans, one or more fructans, lactulose, or any mixture of any two or more thereof.

In certain embodiments, the composition comprises L. rhamnosus HN001 and one or more prebiotics, one or more probiotics, one or more postbiotics, one or more sources of dietary fibre. In certain embodiments, the prebiotic comprises one or more fructooligosaccharides, one or more galactooligosaccharides, inulin, one or more galactans, one or more fructans, lactulose, or any mixture of any two or more thereof.

Without wishing to be bound by theory, it is believed that co-culture and/or co-administration of two or more strains of lactic acid bacteria, such as three strains of lactic acid bacteria, can reduce the incidence of culture failure due to infection by bacteriophages. Accordingly, in certain embodiments, the composition comprises L. rhamnosus HN001 and one or more other strain of lactic acid bacteria, preferably two or more other strains of lactic acid bacteria. In other embodiments, the composition comprising L. rhamnosus HN001 is administered simultaneously or sequentially with one or more other compositions comprising one or more other strains of lactic acid bacteria, preferably two or more other strains of lactic acid bacteria.

It will be appreciated that different compositions of the invention may be formulated with a view to administration to a particular monogastric subject group. For example, the formulation of a composition suitable to be administered to pigs may differ to that suitable to be administered to a different animal, such as horses. It should also be appreciated that compositions of the invention may be formulated differently to be suitable to be administered to monogastric animals of different ages. For example, the formulation of a composition suitable to be administered to piglets or foals may differ to that suitable to be administered to adult pigs. In certain embodiments, a first composition may be formulated for administration to young animals, such as pre-weaning animals, in an initial dosing regimen, and a second composition may be formulated for administration to the same animals in a maintenance dosing regimen. In some embodiments, the first composition is formulated for pre-weaning animals and the second composition is formulated for post-weaning animals.

### Preparation of L. rhamnosus HN001

Direct-fed microbials (DFMs) and their use in methods to modulate ruminal function and improve monogastric performance is known in the art, as are methods for their production.

Briefly, L. rhamnosus HN001 can be cultured using conventional liquid or solid fermentation techniques. In at least one embodiment, the strain is grown in a liquid nutrient broth, to a level at which the highest number of spores are formed. The strain is produced by fermenting the bacterial strain, which can be started by scaling-up a seed culture. This involves repeatedly and aseptically transferring the culture to a larger and larger volume to serve as the inoculum for the fermentation, which can be carried out in large stainless-steel fermenters in medium containing proteins, carbohydrates, and minerals necessary for optimal growth. Non-limiting exemplary media are MRS or TSB. However, other media can also be used. After the inoculum is added to the fermentation vessel, the temperature and agitation are controlled to allow maximum growth. Once the culture reaches a maximum population density, the culture is harvested by separating the cells from the fermentation medium. This is commonly done by centrifugation.

In one embodiment, to prepare the L. rhamnosus HN001 strain, the L. rhamnosus HN001 strain is fermented to a 1×10⁸ CFU/ml to about 1×10⁹ CFU/ml level. The bacteria are harvested by centrifugation, and the supernatant is removed. The pelleted bacteria can then be used to produce a DFM. In at least some embodiments, the pelleted bacteria are freeze-dried and then used to form a DFM. However, it is not necessary to freeze-dry the strain before using them. The strain can also be used with or without preservatives, and in concentrated, unconcentrated, or diluted form.

The count of the culture can then be determined. CFU or colony forming unit is the viable cell count of a sample resulting from standard microbiological plating methods. The term is derived from the fact that a single cell when plated on appropriate medium will grow and become a viable colony in the agar medium.

Since multiple cells may give rise to one visible colony, the term colony forming unit is a more useful unit measurement than cell number.

In another embodiment, the L. rhamnosus HN001 is cultured using a milk-based carrier, such as thermalized milk, to create a fermented yoghurt-style composition. Methods to create such fermented yoghurt-style compositions are well known in the art, and may include, for example, using a warm water bath or other heating means to incubate the milk at a suitable temperature until a sufficient cell density is reached, such as over 12 hours. In one embodiment, the temperature is 25-30° C. Optionally, the milk may include other additives to promote bacterial growth, such as yeast extract. In certain embodiments, the culturing takes place on-site, such as on the farm where the probiotic feed supplementation is to take place.

### EXAMPLES

### 1. Example 1—Plate-based Screen of Lacticaseibacillus rhamnosus HN001 Against an Indicator Methanogen Strain

### 1.1 Materials and Methods

### 1.1.1 Methanogen Culture

The inoculum of an indicator methanogen strain (Methanobrevibacter boviskoreani JH1) for seeding the plate assay was grown in 9 mL BY medium (Joblin, 2005) supplemented with 0.2 mL of 3 M sodium formate, 0.2 mL of 10 M ethanol, 0.1 mL of Vitamin Solution (Janssen et al., 1997) and 0.1 mL of Coenzyme M Solution (Sigma Aldrich, 0.1 M) by syringe using anaerobic techniques. The head spaces of the tubes were pumped with pressurized O₂-free CO₂ to 180 kPa and the tubes were incubated at 39° C. without shaking until visible turbidity appeared after 3 to 5 days. Methane produced by the methanogen strain was measured by removing a sample of the headspace gases by syringe and injecting on to a gas chromatograph (GC; Aerograph Corporation, USA) equipped with a Thermal Conductivity Detector (TCD) and using nitrogen as the carrier gas. The gas inside the tubes was released by venting with a sterile needle to prevent over pressurization. The cultures were routinely observed via wet mounts under fluorescence microscopy, and the methanogen strain appears as short ovoid-shaped rods that fluoresce green under ultraviolet (UV) illumination. The cultures were also checked for contamination by inoculating a sample of the culture into 9 mL BY media supplemented with 0.1 mL of 0.5 M glucose and incubating at 39° C. for one day. If no turbidity was seen after 1 day, then the culture was considered uncontaminated. Further verification was conducted from time to time by extracting the genomic DNA from the methanogen strain culture and PCR amplifying the 16S rRNA gene, using both the conventional bacterial 16S primers (27f-GAGTTTGATCMTGGCTCAG, 1492r-GGYTACCTTGTTACGACTT) and the archaeal-specific 16S primers (915af-AGGAATTGGCGGGGGAGCAC, 1386r-GCGGTGTGTGCAAGGAGC).

The presence of a band with the archaeal primer set and the absence of a band with the bacterial primer set, and the sequencing results of PCR products, were used to validate culture purity.

### 1.1.2 Preparation of Strains to be Tested

Cultures of L. rhamnosus HN001, and control strains (L. plantarum ATCC 8014, L. bulgaricus ATCC 11842) were grown overnight in MRS broth (Sigma-Aldrich) at 39° C. The optical density at 600 nm (OD₆₀₀) was measured for each culture, and a sample of each was serially diluted through MRS medium and the dilutions plated onto MRS agar plates to determine viable counts. For each bacterial culture to be tested, 3 mL of the overnight culture was removed anaerobically from the tube using a 5 mL disposable syringe fitted with a 21G needle, along with 1 mL of CO₂ from the culture headspace. The used needle on the syringe was replaced with a Millex 33 mm filter (0.22 μm; Merck Millipore) and a fresh 21G needle attached. The 1 mL of CO₂ was pushed out through the filter and new needle to flush them with the CO₂ from the headspace and make them anaerobic. The needle was then inserted into a sterile, CO₂-flushed Hungate tube and the culture filtrate was pushed through the filter into the tube. Once prepared, the filtrates in the Hungate tubes were placed into an anaerobic chamber. All of the assay components were assembled in the anaerobic chamber as indicated in Table 1. Multiwell, 96 well plates were then placed into an AnaeroPack 2.5L Rectangular Jar along with an Anaeropack-Anaero Anaerobic Gas Generator. The lid was sealed, the jar removed from the anaerobic chamber and incubated at 39° C. The plate was observed daily through the transparent jar, and when the methanogen strain control had visible turbidity, the plate was removed from the jar and the optical density (OD₆₀₀) was recorded after 5 seconds shaking in a SpectraMax plate reader. The absorbance readings of the media control wells were subtracted as background, and the % inhibition of methanogen strain growth caused by the filtrate samples, relative to the positive growth control wells, was calculated.

TABLE 1
Plate setup for the Methanogen bioassay.

| | Volumes added (final concentration) in 250 μL per well | | | | |
|---|---|---|---|---|---|
| | | | L. plantarum/L. | | |
| | | | bulgaricus | | L. rhamnosus |
| | Media | Methanogen | filtrate | | HN001 |
| Component | control | alone control | control | Nisin | filtrate |
| Media | 75 μL | 75 μL | 0 | 65 μL | 0 |
| (MRS) | (30% v/v) | (30% v/v) | | | |
| Nisin (from | 0 | 0 | 0 | 10 μL | 0 |
| Lactococcus | | | | (12 μM) | |
| lactis; Sigma | | | | | |
| Aldrich; | | | | | |
| 1 mg/ml, | | | | | |
| 300 μM) | | | | | |
| Filtered | 0 | 0 | 75 μL | 0 | 75 μL |
| supernatant | | | (30% v/v) | | (30% v/v) |
| Phosphate | 5 μL | 5 μL | 5 μL | 5 μL | 5 μL |
| buffer (0.5M | (20 mM) | (20 mM) | (20 mM) | (20 mM) | (20 mM) |
| K₂HPO₄, 0.5M | | | | | |
| KH₂PO₄, pH 7) | | | | | |
| Methanogen | 0 | 10 μL | 10 μL | 10 μL | 10 μL |
| inoculum | | (4% v/v) | (4% v/v) | (4% v/v) | (4% v/v) |
| BY medium | 155 μL | 145 μL | 145 μL | 145 μL | 145 μL |
| Ethanol | 5 μL | 5 μL | 5 μL | 5 μL | 5 μL |
| (10M) | (200 mM) | (200 mM) | (200 mM) | (200 mM) | (200 mM) |
| Vitamin/COM | 5 μL | 5 μL | 5 μL | 5 μL | 5 μL |
| solution | (2% v/v) | (2% v/v) | (2% v/v) | (2% v/v) | (2% v/v) |
| Formate | 5 μL | 5 μL | 5 μL | 5 μL | 5 μL |
| (3M) | (60 mM) | (60 mM) | (60 mM) | (60 mM) | (60 mM) |
| TOTAL | 250 μL | 250 μL | 250 μL | 250 μL | 250 μL |

### 1.2 Results

The L. rhamnosus HN001 culture grew well on MRS broth, attaining an OD₆₀₀ of 4.97 after 16 hours growth. Viable counts from plating of dilutions of the culture onto MRS plates indicated 4.8×10⁹ CFU·mL⁻¹ of culture. These growth parameters were similar to the control strains L. plantarum 8014 and L. bulgaricus 11842, although L. plantarum 8014 had a lower viable count. Filtrates from the test strains were included in the methanogen bioassay, and the plate was incubated at 39° C. for 5 days before being removed from the jar and the OD₆₀₀ of the wells recorded. The readings of the test wells were compared with those of the methanogen strain without any treatment and the % inhibition of growth is shown in Table 2.

TABLE 2
L. rhamnosus HN001 culture filtrate screening
against the indicator methanogen strain.

| | OD₆₀₀ of | | % | | | |
|---|---|---|---|---|---|---|
| | test | | methanogen | | inhibition | |
| Treatment | culture | CFU/mL | growth* | Std Dev | % | P |
| L. plantarum | 4.78 | 2.8E+08 | 91.1 | 3.9 | 8.9 | <0.001 |
| 8014 | | | | | | |
| L. bulgaricus | 4.85 | 2.1E+09 | 106.9 | 11.0 | −6.9 | 0.06 |
| 11842 | | | | | | |
| L. rhamnosus | 4.97 | 4.8E+09 | 77.1 | 14.8 | 22.9 | <0.001 |
| HN001 | | | | | | |
| Nisin (4 μM) | NA | NA | 12.2 | 8.0 | 87.8 | <0.001 |

*% inhibition was calculated from the mean of OD₆₀₀ readings from 16 wells per treatment.

The L. rhamnosus HN001 filtrate significantly reduced the growth of the methanogen strain, on average by nearly 23%. This growth inhibition was higher than seen with either of the control strains, L. plantarum 8014 or L. bulgaricus 11842, which reduced growth by around 9% or had no effect, respectively. The L. rhamnosus HN001 filtrate showed inhibitory activity approximately 25% of the 4 μM nisin control treatment.

### 1.3 Discussion

The methanogen inhibitory activity observed in the testing of culture supernatants of L. rhamnosus HN001 was greater than the inhibition seen with the two control strains.

M. boviskoreani JH1 was used as the indicator strain because the most prevalent methanogenic archea in the gastrointestinal tract of multiple monogastric species belong to the genus Methanobrevibacter (Misiukiewicz, A et al. (2021).

### 1.4 Conclusion

The screening of L. rhamnosus HN001 culture supernatant in a plate-based assay demonstrated an inhibition of the indicator methanogen strain. In relation to control LAB strains, this inhibitory activity was greater than that observed with L. plantarum 8014 or L. bulgaricus 11842 but less than a purified nisin control.

### 2. Example 2—The Effect of Lacticaseibacillus rhamnosus HN001 on Microbiota Composition

### 2.1 Materials and Methods

### 2.1.1 Animals

This study was carried out in strict accordance with the NZ Animal Welfare Act 1999, and under consideration by the AgResearch Limited (Grasslands) Animal Ethics Committee (Ethics Approval No.: 13982).

Twenty-four male large white cross ten-day old piglets were obtained from a commercial farm in the Manawatu-Wanganui region of New Zealand. All piglets were housed in custom cages constructed to allow animals to see, hear, and smell adjacent piglets, but still preventing physical contact (Mudd et al., 2017). On arrival at the animal facility (day 1), the piglets were pair-housed for two nights. The piglets were exclusively fed reconstituted infant formula (control formula; Fonterra Nutritional Base+DCL 100 at 5 g/L of formula) two hours post-arrival and then every four hours after that feed. From day 2 to 24 the piglets were individually housed. During this period, the piglets were let out into a shared pen and allowed to physically interact for an hour of social time each day.

From day 3 to day 24, the piglets were assigned to one of three treatment groups; 8 receiving control formula; 8 receiving control formula supplemented with 2.43×10⁵ CFU/ml of Lacticaseibacillus rhamnosus HN001 (HN001™ Low); and 8 receiving control formula supplemented with 1.43×10⁶ CFU/ml of L. rhamnosus HN001 (HN001™ High). All formulas were dispensed using an automated system programmed to offer the formula every 2 hours with automatic measurement of refusals. The piglet caging and automated feeding system was designed and manufactured by ShapeMaster (Ogden, IL, USA).

At the end of the study, the piglets were euthanised by sedation using a cocktail of Tiletamine hydrochloride, Zolazepam Hydrochloride, Xylazine, and ketamine hydrochloride (final solution 50 mg/ml of each drug), followed by intracardiac puncture with sodium pentobarbitone (300 mg/ml). Caecal contents were collected and stored at −80° C. for microbiome analyses.

### 2.1.2 Microbiome Analysis

Metagenomic DNA was extracted from caecal contents using Macherey Nagel NucleoSpin Soil kits (Duren, Germany) following manufacturer's instructions with the addition of bead beating on a BioSpec Mini-Beadbeater 96 (Bartlesville, OK, USA) set to 4 min.

A total amount of 1 μg of metagenomic DNA per sample was used as input material for the DNA sample preparations. Sequencing libraries were generated using NEBNext® Ultra DNA Library Prep Kit for Illumina (NEB, USA) following manufacturer's recommendations and index codes were added to attribute sequences to each sample. Briefly, the DNA sample was fragmented by sonication to a size of 300 bp, then DNA fragments were end-polished, A-tailed, and ligated with the full-length adaptor for Illumina sequencing with further PCR amplification. The PCR products were purified (AMPure XP system) and libraries were analysed for size distribution by Agilent2100 Bioanalyzer and quantified using real-time PCR. Clustering of index-coded samples was performed on a cBot Cluster Generation System according to the manufacturer's instructions. After cluster generation, the library preparations were sequenced on an Illumina HiSeq X platform and 150 bp paired-end reads were generated.

Read pairs were joined using PEAR version 0.9.6 (Zhang et al., 2014) with default settings. Read pairs that did not join were concatenated with a spacer consisting of a string of N's using the “fuse” function from the BBMAP package version 38.22-0 (Bushnell, 2014). Evaluation and detection of host reads were done using the bbduk.sh function from the BBMAP package version 38.22-0 [PM2], a k-mer based filter, with the pig genome (Sscrofa 11.1 release 96) as reference. Metaxa2 version 2.1.3a (Bengtsson-Palme et al., 2015) was used to assign taxonomy to the reads from SSU ribosomal DNA using the Silva 128 database (Quast et al., 2013). The “blastx” function of DIAMOND version 0.9.22 (Buchfink et al., 2015) was used to map the reads against the “nr” NCBI database [PM6]. Megan version 6 ultimate edition (Huson et al., 2016) was used to assign putative functions to the DIAMOND alignment files against the SEED Subsystems database (Overbeek et al., 2005).

Comparisons of overall community compositions were performed using permutational multivariate analysis of variance (PERMANOVA) of distance matrices, implemented through the adonis function from the vegan package (Dixon, 2003) for R. Differences in the relative abundances of individual taxa and gene functions were analysed by permutation ANOVA using the aovp function from the ImPerm package (Wheeler and Torchiano, 2016) for R. Resulting P values were adjusted for multiple testing using the False Discovery Rate (FDR).

### 2.2 Results

2.2.1 Lacticaseibacillus rhamnosus Relative Abundances

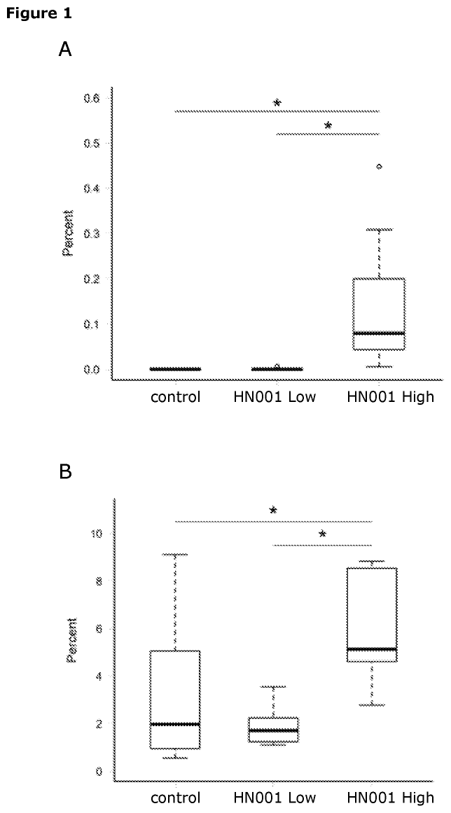

Analysis of sequence data at the species level showed supplementation with L. rhamnosus HN001 increased the relative abundance of L. rhamnosus in the caecal community at the higher dose (p<0.001 and p=0.007, respectively), compared to the Control and HN001™ Low groups (FIG. 1A). Because of the uncertainty with classification to the species level (Peabody et al., 2015; Johnson et al., 2019), we also considered the relative abundance of taxa classified as uncultured and unclassified Lactobacillus. When these were combined with sequences identified as L. rhamnosus, we saw a similar pattern in the caecum where the relative abundance of these combined sequences was higher in the HN001™ High group (p=0.018; FIG. 1C).

### 2.2.2 Caecal Microbiome

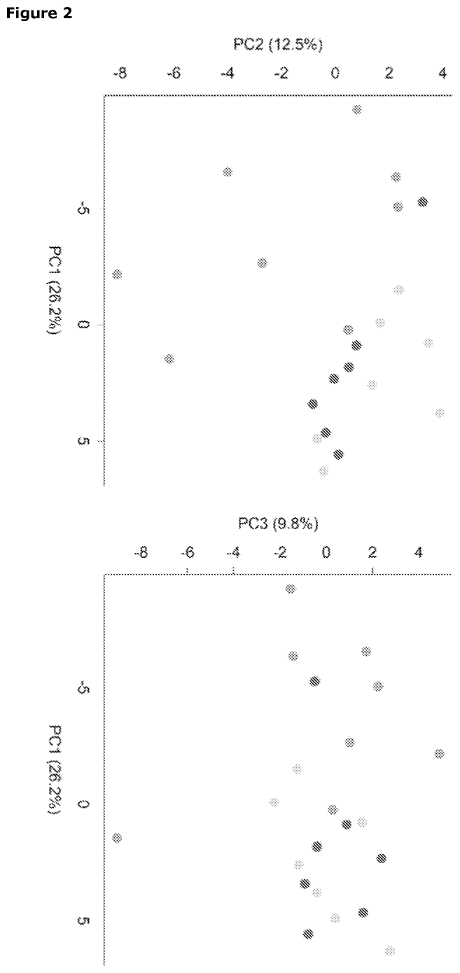

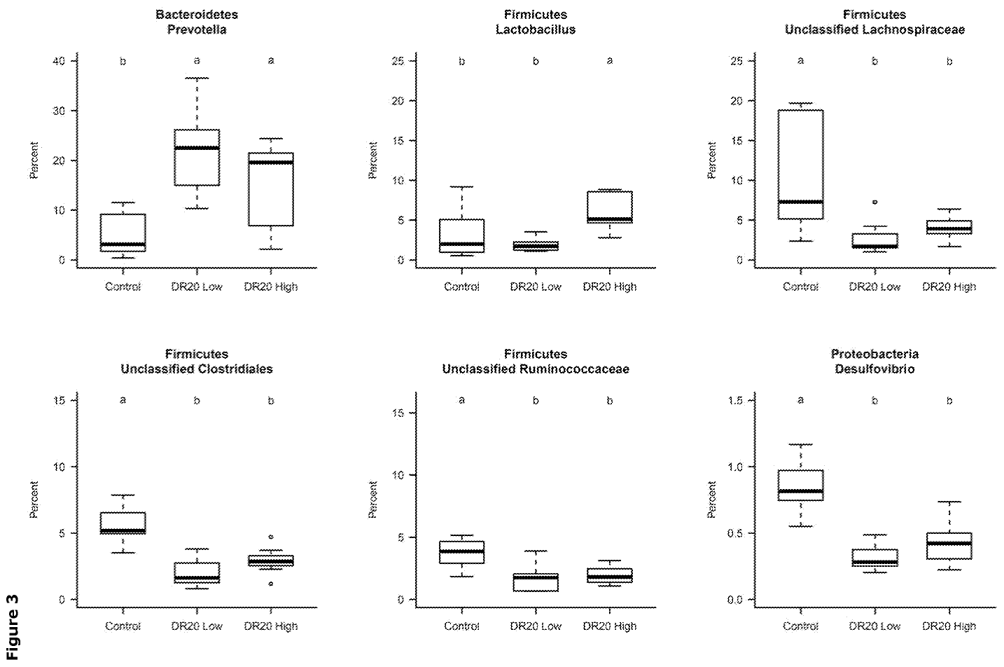

Supplementation with L. rhamnosus HN001 also led to dramatic changes in the overall caecal microbiome composition (FIG. 2, permutation MANOVA p=0.001; Table 1; FIG. 3). Piglets in the low and high HN001™ groups had increased relative abundance of Bacteriodetes compared the Controls (FDR=0.018), along with a concomitant decrease in the Firmicutes (FDR=0.007). L. rhamnosus HN001 also decreased the abundance of Verrucomicrobia (FDR=0.039) and the archaeal phylum Euryarchaeota (FDR=0.019).

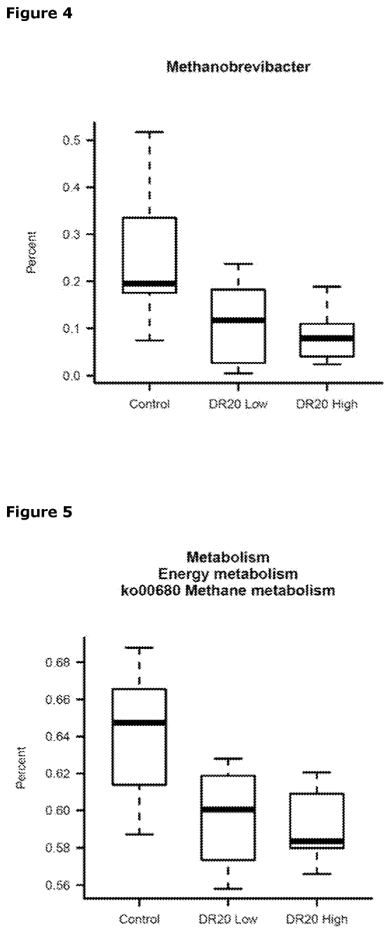

The broad changes in community composition were accompanied by significant differences that could be detected at lower taxonomic levels. These included the Lactobacillus genus as a whole, which was significantly higher in piglets fed the high dose of HN001™ compared to the Control and low HN001™ group (FDR=0.045) and Prevotella, which were higher in piglets fed either dose of HN001™ compared to Control (FDR=0.027). The change in Prevotella was particularly notable as they underwent a major fold change while also comprising a substantial proportion of the community (Control 5%±1.5; HN001™ Low 21.7%±3.5; HN001™ High 14.7%±3.5; mean %±SEM). Other genera that differed included the Lachnospiraceae, Peptostreptococcaceae, and Ruminococcaceae, which were all decreased by HN001™ supplementation (FDR<0.05). Desulfovibrio, a sulphate-reducing Proteobacteria, and Methanobrevibacter, a methane-producing archaea, were also decreased by feeding HN001™ (FDR<0.001 and FDR<0.05 respectively; FIGS. 3 and 4).

### 2.2.3 Caecal Metagenomic Analysis

Changes to the caecal microbiome were also apparent in by differences in the relative abundances of genes related to a wide range of metabolic processes and pathways. Analysis of genes mapped to the SEED Subsystems database (Table 2) showed 40 level 2 functions (out of 1077 analysed) were significantly different in relative abundance between groups (FDR<0.05). These included genes involved in carbohydrate metabolism; genes annotated to the SEED function Lactate utilization were more relative abundant in the HN001™ Low and HN001™ High group compared to Controls (FDR<0.05), while genes categorised to Pyruvate:ferredoxin oxidoreductase and Methanogenesis were lower in relative abundance for both HN001™ groups compared to Controls (FDR<0.05). Other SEED categories related to methanogens and methane metabolism also differed in relative abundance (FIG. 5); Carbon monoxide induced hydrogenase, H₂:CoM-S-S-HTP oxidoreductase, and Aromatic amino acid interconversions with aryl acids were also lowered in both HN001™ groups compared to Controls (FDR<0.05).

TABLE 3
Microbial taxa| with > 0.5 mean relative abundances in the caecum with significant differences (permutation
ANOVA FDR < 0.05) between piglet fed infant formula supplemented with HN001 and control formula. Values indicate
mean percent ± standard error of means. Means within each microbial taxon without a letter differ significantly.

| Phylum | Family | Genus | Control | HN001 ™ Low | HN001 ™ High | P value | FDR |
|---|---|---|---|---|---|---|---|
| Bacteroidetes | | | 32.89 ± 3.73 ^{b} | 51.21 ± 3.39 ^{a} | 40.95 ± 3.02 ^{a} | 0.0054 | 0.018 |
| Bacteroidetes | Prevotellaceae | | 12.47 ± 2.55 ^{b} | 35.12 ± 4.26 ^{a} | 25.04 ± 4.61 ^{a} | 0.0014 | 0.0288 |
| Bacteroidetes | Prevotellaceae | Prevotella | 5 ± 1.54 ^{b} | 21.67 ± 3.45 ^{a} | 14.68 ± 3.5 ^{a} | 0.0026 | 0.0267 |
| Firmicutes | | | 45.92 ± 4.25 ^{a} | 23.90 ± 2.52 ^{b} | 32.24 ± 2.25 ^{b} | 0.0006 | 0.0072 |
| Firmicutes | Lactobacillaceae | Lactobacillus | 3.22 ± 1.12 ^{b} | 1.91 ± 0.34 ^{b} | 6.17 ± 0.93 ^{a} | 0.0076 | 0.0497 |
| Firmicutes | Lachnospiracea | | 14.82 ± 3.27 ^{a} | 4.49 ± 1.12 ^{b} | 6.01 ± 0.75 ^{b} | 0.009 | 0.0498 |
| Firmicutes | Peotostreptococcaceae | | 0.85 ± 0.15 ^{a} | 0.25 ± 0.06 ^{b} | 0.34 ± 0.11 ^{b} | 0.0032 | 0.0461 |
| Firmicutes | Ruminococcaceae | | 7.11 ± 0.72 ^{a} | 3.52 ± 0.8 ^{b} | 4.18 ± 0.62 ^{b} | 0.004 | 0.0473 |
| Proteobacteria | Desulfovibrionaceae | | 0.89 ± 0.07 ^{a} | 0.34 ± 0.04 ^{b} | 0.44 ± 0.07 ^{b} | <0.0001 | <0.0001 |
| Proteobacteria | Desulfovibrionaceae | Desulfovibrio | 0.85 ± 0.07 ^{a} | 0.32 ± 0.04 ^{b} | 0.43 ± 0.07 ^{b} | <0.0001 | <0.0001 |
| Euryarchaeota | Methanobacteriaceae | Methanobrevibacter | 0.25 ± 0.05 | 0.11 ± 0.04 | 0.08 ± 0.02 | 0.0056 | 0.045 |

TABLE 4
Microbial SEED level 2 functions with significantly different (permutation ANOVA FDR < 0.05) mean relative
abundances from the caecum of piglets fed infant formula containing different doses of HN001. Values indicate
mean percent ± standard error of means. Means within each function without a common letter differ significantly.

| SEED Function (Level 1; Level 2) | Control | HN001 ™ Low | HN001 ™ High | P value | FDR |
|---|---|---|---|---|---|
| Amino Acids and Derivatives; Aromatic amino acid | 0.054 + 0.007^{a} | 0.027 + 0.004^{b} | 0.028 + 0.006^{b} | 0.0014 | 0.049 |
| interconversions with aryl acids; | | | | | |
| Carbohydrates; Lactate utilization; | 0.05 + 0.004^{b} | 0.079 + 0.005^{a} | 0.078 + 0.005^{a} | <0.0001 | <0.0001 |
| Carbohydrates; Methanogenesis; | 0.015 + 0.0024^{a} | 0.007 + 0.001^{b} | 0.007 + 0.001^{b} | 0.0004 | 0.0284 |
| Carbohydrates; Pyruvate: ferredoxin oxidoreductase; | 0.109 + 0.005^{a} | 0.089 + 0.004^{b} | 0.083 + 0.004^{b} | 0.0014 | 10.049 |

### 2.3 Discussion

In this example we show that supplementation with L. rhamnosus HN001 can have dramatic effects on the microbiome of the caecum in a piglet model, with broad changes in relative abundance of the dominant taxa.

Supplementation with L. rhamnosus HN001 at the highest dose led to increased proportions of L. rhamnosus in the caecum. Similarly, the relative abundance of the Lactobacillus genus overall was increased in the HN001™ High group.

While caecal Lactobacillus proportions increased from around 3% in control piglets to around 6% in the HN001™ High group, greater changes, both in terms of fold change and relative abundance, were observed in a wide range of taxa. Bacteriodetes relative abundance in the caecum was increased by both low and high doses of L. rhamnosus HN001, which was accompanied by a concomitant decrease in the Firmicutes. Within the Bacteroidetes, the most prominent change occurred in Prevotella, which increased over 3-fold on supplementation with L. rhamnosus HN001. Increased Prevotella has been associated with improved glucose response (Kovatcheva-Datchary et al., 2015) and reduction in body fat (Hjorth et al., 2019). Prevotella is also an important member of the gut community that contributes to polysaccharide breakdown and SCFA production (Precup and Vodnar, 2019). HN001-induced increases in the level of Prevotella can thus result in increased levels of SCFA, which the applicant considers does act as an increased energy source driving enhanced growth or increased productivity. The applicant considers that this will also improves body composition and increases food efficiency.

Other changes in the caecal microbiome included the hydrogen-using microbes Desulfovibrio and Methanobrevibacter, both of which were significantly decreased in both HN001™ groups compared to controls. Desulfovibrio is a prominent sulfate-reducing bacterium in the human colon metabolises H₂ to form H₂S while Methanobrevibacter converts CO₂ and H₂ to form methane (Smith et al., 2019). For both microbes, an important limiting factor for this conversion is the concentration of H₂, which in the gut is mainly produced by microbial fermentation of carbohydrates (Flint et al., 2012). Increased lactate production from lactic acid bacteria may promote the activity of lactate-utilising microbes and shifting microbial fermentation towards pathways that reduce the formation of hydrogen (Doyle et al., 2019). We found genes involved in lactate utilisation were increased in caecum of piglets supplemented with L. rhamnosus HN001. Furthermore, free hydrogen appears to be produced primarily by the Firmicutes (Carbonero et al., 2012), which were significantly reduced in piglets supplemented with L. rhamnosus HN001.

Other differences that indicate pathways involve in carbohydrate fermentation and hydrogen utilisation are altered by L. rhamnosus HN001 include changes in gene abundances related to methanogen metabolism, such as the SEED functions Carbon monoxide induced hydrogenase, H2:CoM-S-S-HTP oxidoreductase, and Aromatic amino acid interconversions with aryl acids. Carbon monoxide induced hydrogenase genes can be used by methanogens to use methyl groups as a substrate (Morishita et al., 1999), while reduction of heterodisulfide (CoM-S-S-HTP) is a key energy metabolism pathway in methanogenic Archaea (LeBlanc et al., 2011). Interestingly, the SEED function Aromatic amino acid interconversions with aryl acids, an important pathway used by methanogens to transform aromatic amino acids to aryl amino acids (Soto-Martin et al., 2020), was also reduced in both HN001™ groups compared to Controls.

### 2.4 Conclusions

This example shows that feed supplementation with L. rhamnosus HN001 can have dramatic effects on the developing caecal microbiome in a piglet model. The changes observed could not be explained simply by the expansion of the Lactobacillus genus as the magnitude of differences in other taxa were greater than the magnitude of the increase in Lactobacillus in the L. rhamnosus HN001 supplemented groups. The differences in taxonomic composition and relative abundances of gene functions in the caecum suggests L. rhamnosus HN001-induced changes in the microbiome included alterations in carbohydrate and hydrogen metabolism. Based on these results, the applicant considers that L. rhamnosus HN001 has a specific inhibitory effect on methanogens in the gastrointestinal tract of monogastric animals.

### 3. Example 3—The Effect of Lacticaseibacillus rhamnosus HN001 on Gut Tissue Transcription

### 3.1 Materials and Methods

### 3.1.1 Animals

Piglets were reared and treated with control, HN001™ Low, and HN001™ High formula as described in section 2.1.1 of Example 2.

### 3.1.2 Piglet Gut Tissue Transcriptome

Piglets were euthanised as described in section 2.1.1 of Example 2. Tissue samples from the caecum were collected into RNAlater (Qiagen, Hilden, Germany) and at −80° C. for gene expression analysis.

Gene expression profiles from caecal tissue samples were analysed by RNAseq. Total RNA was extracted from using RNeasy Mini Kits (Qiagen). Total RNA quality and quantity were determined using an Agilent 2100 Bioanalyzer Instrument (Agilent, Santa Clara, CA, USA) and Nanodrop (Thermo Fisher, Waltham, MA, USA), and sample quality was also assessed using agarose gel electrophoresis. Samples that passed the RNA integrity number (RIN) threshold of 6.5 were submitted for sequencing. Strand-specific cDNA libraries were prepared using NEBNext® Ultra Directional RNA Library Prep Kit for Illumina® (Illumina, San Diego, CA, USA) according to the manufacturer's guidelines. Libraries were size selected for 250-300 bp fragments and sequenced using the Novaseq 6000 platform (Illumina) to produce 150 bp paired-end sequences. The reads were quality trimmed using Trimmomatic 0.36 (Bolger et al., 2014) in paired-end mode using the following parameters; LEADING:3 TRAILING:3 SLIDINGWINDOW:4:15 MINLEN:36. Read pairs that passed quality trimming were mapped against the genome (Sscrofa 11.1 release 96) using STAR (Dobin et al., 2013). uniquely mapped read pairs were summed for each gene and analysed using the EdgeR package (Robinson et al., 2010) in R. The resulting counts were analysed using a likelihood ratio generalized linear model, with genes that had >1.5-fold difference (i.e. log fold change >|0.58|) and FDR<0.05 considered differentially expressed. Gene expression profiles were also analysed by gene set enrichment analysis (GSEA) using the mroast function from limma (Smyth, 2004) and KEGG pathways (Kanehisa and Goto, 2000; Carlson, 2016) as gene sets. GSEA involves the analysis of the collective expression of groups of genes treated as a unit rather than as individual genes.

### 3.2 Results

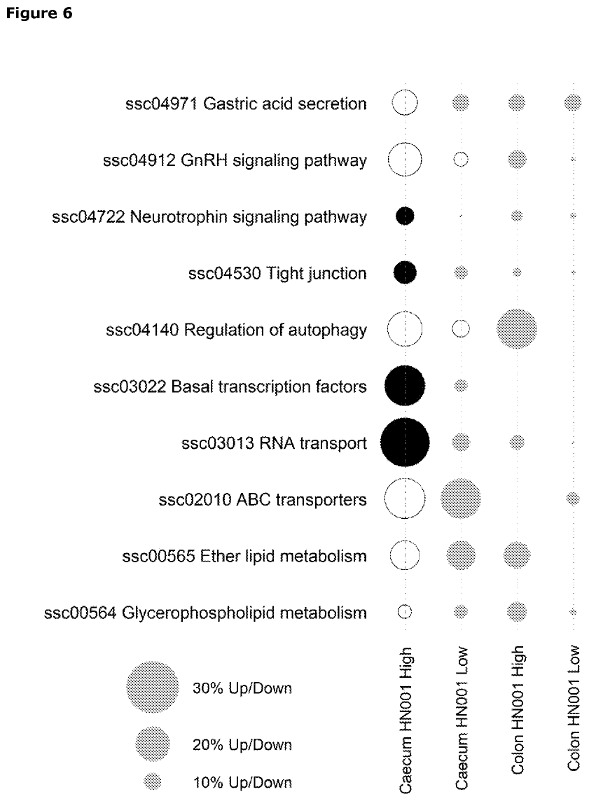

GSEA identified 10 KEGG pathways that were differentially expressed (p<0.05) in the caecum of piglets in the HN001™ High group, and of these two pathways were also differentially expressed in the HN001™ Low group (FIG. 6).

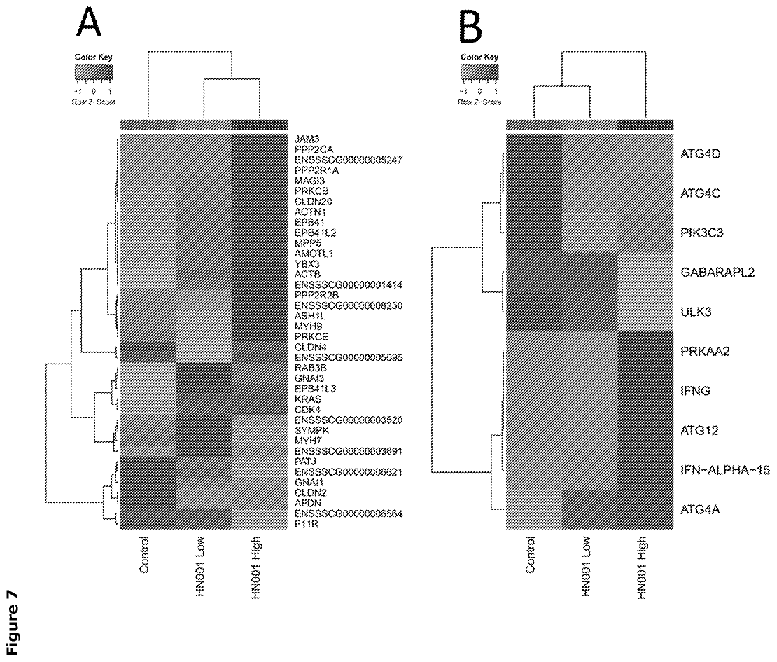

Analysis of the collective gene expression profiles in each of these KEGG pathways by permutation ANOVA also confirmed the overall significant changes in expression patterns in four pathways between the HN001™ High and the Control groups; Tight junction (ssc04530), Basal transcription factors (ssc03022), and RNA transport (ssc03013) pathways were more highly expressed in the HN001™ High group (p=0.005, 0.034, and 0.008, respectively), whereas the Regulation of autophagy (ssc04140) pathway showed higher expression in the Control group (p=0.008). Although expression of KEGG pathways were not significantly altered at the low dose of HN001™, with the exception of the GnRH signalling pathway (ssc04912) and Regulation of autophagy (ssc04140) pathways, expression profile patterns were generally intermediate between controls and the HN001™ High dose piglets (FIG. 7).

### 3.3 Discussion

Associated with the change in caecal microbiota composition shown in Example 2, we showed HN001™ also altered the caecal tissue transcriptome compared to controls.

In addition to the extensive alterations in the microbial community composition of the caecum, supplementation with HN001™ also led to changes in gene expression profiles in several key pathways. Overall expression of genes involved in tight junction formation and activity were more highly expressed in the caecum of piglets receiving the higher dose of HN001™, compared to controls. Tight junction proteins are important molecules that strongly influence intestinal barrier integrity. Intestinal barrier integrity is essential for efficient nutrient absorption and protecting the host from invading pathogens, toxins and antigens. Perturbations in this barrier can lead to inflammation and serious disorders in the gastrointestinal tract and other parts of the body (Groschwitz and Hogan, 2009). Previous studies have shown that HN001™ can beneficially enhance intestinal barrier integrity, both in cell models (Anderson et al., 2010) and animal models (Kawasaki and Kawai, 2014). Treatment with HN001™ in animal models have also been shown to decrease the severity of necrotizing enterocolitis, characterised by extensive destruction of the intestinal epithelial cell layer (Good et al., 2014). In this instance, the protective effects of HN001™ was modulated by the activation of Toll-like receptor 9 (Good et al., 2014), a receptor for microbial DNA expressed in immune system cells including dendritic cells, and other antigen presenting cells (Kawasaki and Kawai, 2014). Both intestinal barrier integrity and immune status are also both important factors associated with feed efficiency (McCormack et al., 2019).

Related to the effects on tight junction gene expression, HN001™ also altered expression in pathways related to neutrophin signalling and autophagy. The neurotrophic factor glial cell-derived neurotrophic factor (GDNF) has been shown to attenuate inflammation by increasing expression of tight junction proteins in a mouse model (Reinshagen et al., 2000). Increased expression of another neurotrophic factor, brain-derived neurotrophic factor (BDNF), has also been shown to suppress autophagy in mice (Nikoletopoulou et al., 2017). In this example, expression of neutrophin signalling pathways in the caecal tissue was increased by HN001™ at the high dose, while expression of autophagy pathways was decreased. Autophagy is a cellular process controlling the ordered removal of dysfunctional cells, which plays a major role in the regulation of inflammation (Matsuzawa-Ishimoto et al., 2018). Direct links between autophagy proteins and tight junction integrity has also been demonstrated in previous studies. For example, autophagy-related protein-6 (ATG6) has been shown to disrupt tight junction integrity in a cell model by promoting the endocytosis of the tight junction protein occludin (Wong et al., 2019), while in a rat model of intestinal inflammation, decreased autophagy was associated with upregulation of claudin-2 (Huang et al., 2019), another tight junction protein. These studies and our results highlight how inflammation, intestinal barrier function and the intestinal microbes, whether resident or introduced, are interconnected and can improve for example, feed efficiency.

### 3.4 Conclusions

Concomitant with alterations in the caecal microbiome described in Example 2, host gene expression profiles in the caecal tissue were also impacted by HN001™ supplementation. Changes in gene expression suggest HN001™ improves intestinal barrier integrity and decreased inflammation. This work indicates HN001™ can influence the microbiome and host physiology, with changes that that the applicant considers will be beneficial to the host such as improved nutrient absorption, reduced inflammation and improved feed efficiency.

### 4. Example 4—The Effect of Lacticaseibacillus rhamnosus HN001 on Piglet Weight

### 4.1 Materials and Methods

The experimental protocol for a pig probiotic trial using freeze dry probiotic product (Fonterra) was approved by the AgResearch Grasslands Animal Care and Ethics Committee (approval number 15323).

A total of 16 piglets were enrolled in the trial at 3 days of age. The piglets were weighed and assigned at random to one of the 2 treatment groups (n=8): HN001™, receiving 5×10¹⁰ CFU/d of the probiotic organism Lactobacillus rhamnosus HN001™; and the Control group, without LAB feeding. The piglets were placed individually into crates, each fitted with a heating-pad, an automatic milk feeder and free access to water via a water bowl. The piglets remained in these crates except during the time when the crates were being cleaned when they were kept is a large communal open pen where they could interact and play for at least 2 h/d. During the first 5 weeks, the piglets were fed exclusively on milk (reconstituted from milk powder). To feed the piglets, the amount of milk required for 8 piglets was prepared and 1 sachet of freeze dried (FD) HN001 containing the required dose for 8 animals was added to the milk. The milk was placed into the automatic milk feeder connected to an electronic system allowing regular dispensing of milk over a 24 h period. Frozen ice packs were placed around the feeder reservoirs to keep the milk cool and prevent microbial growth. Pigs were weighed every third day.

At 5 weeks of age, solid food was introduced into the diet in the form of Little Pig Tucker pellets (NRM Feeds, NZ), and the piglets were weaned by slowly decreasing the amount of milk offered, (keeping the probiotic dose constant) until week 8 when only pellets were offered twice daily (morning; afternoon). Water was freely available until the end of the trial. During this period at 7 weeks of age the piglets (˜20 kg Live weight; LWT) were moved to larger pens in a covered barn. Each pen had a raised wooden sleeping area with a heat pad, a feed trough and water available via a self-activated nipple. Once the piglets had transitioned completely to the pellet diet, the amount of HN001™ required for 8 pigs was reconstituted in a small amount of water (200 mL) and mixed evenly into ˜1.6 kg of pellets. The pellets containing the HN001™ were then evenly divided into 8 aliquots (200 g/pig) and fed to the appropriate pigs. The Control animals received the same quantity of pellets treated with water only. These pellet+treatment mixtures were provided as the first feed in the morning when pigs were hungry to ensure the entire LAB dose was consumed each day. Once the pellet+treatment mixtures were eaten, the main meal of dry pellets was topped up for the morning feed. The pigs were fed ad libitum pellets from 8 weeks of age until the end of the trial at 19 weeks.

The piglets suffered an episode of rotavirus infection during Week 4 of the trial and the animals were given electrolyte therapy and Scourban Plus (Bayer NZ). A second round of rotavirus infection occurred at Week 9 of the trial after moving the pigs to the larger pens. The pigs were again treated with electrolyte therapy and all animals recovered well. At Week 14, pig #9 (HN001 treatment group) presented a foot injury which required oral antibiotic treatment (Vet-Tet 20; oxytetracycline; 15 g per day for 5 d) and anti-inflammatory treatment (Metacam).

### 4.2 Results

This Example showed that supplementation with L. rhamnosus HN001™ did not significantly affect pig weight (Table 3).

TABLE 3
Effect of bacterial strains on pig weight. Values
indicate mean weight ± standard deviation.

| | Average pig weight (kg) | | |
|---|---|---|---|
| | Control treatment | HN001 ™ | Student T |
| Age (days) | (n = 8) | (n = 8) | test p value |
| 47 | 17.7 ± 1.2 | 17.4 ± 1.9 | 0.77 |
| 51 | 20.8 ± 1 | 20.6 ± 1.6 | 0.73 |
| 53 | 21.9 ± 1 | 21.6 ± 2.1 | 0.78 |
| 58 | 25.2 ± 1.4 | 25.1 ± 2 | 0.95 |
| 61 | 27.8 ± 1.8 | 28.1 ± 2.1 | 0.77 |
| 72 | 35.2 ± 2.3 | 35.1 ± 3.8 | 0.97 |
| 75 | 38.5 ± 1.7 | 37.8 ± 3.5 | 0.62 |
| 79 | 41.9 ± 2.3 | 41.4 ± 4.1 | 0.78 |
| 82 | 45.3 ± 3.8 | 45.4 ± 4.9 | 0.96 |
| 87 | 49.8 ± 3.5 | 49.5 ± 4.8 | 0.91 |
| 90 | 53.8 ± 3 | 52.5 ± 5.7 | 0.62 |
| 94 | 56 ± 3.1 | 55.1 ± 5 | 0.68 |
| 98 | 63.3 ± 4.2 | 61.8 ± 6 | 0.60 |
| 104 | 73.5 ± 5.1 | 72.1 ± 7.2 | 0.69 |
| 111 | 82.8 ± 4.6 | 79.7 ± 8 | 0.39 |
| 122 | 100.1 ± 5.2 | 93.6 ± 7.7 | 0.10 |

Table 3 and

[symbol]

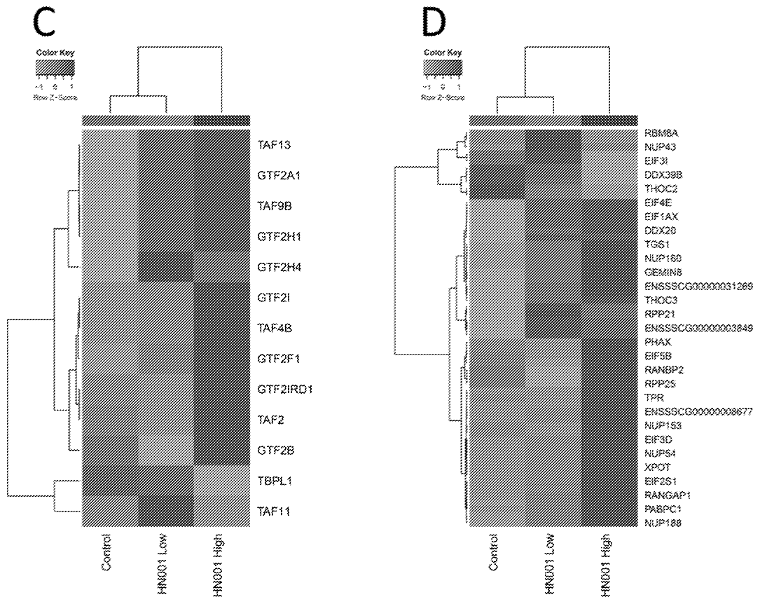

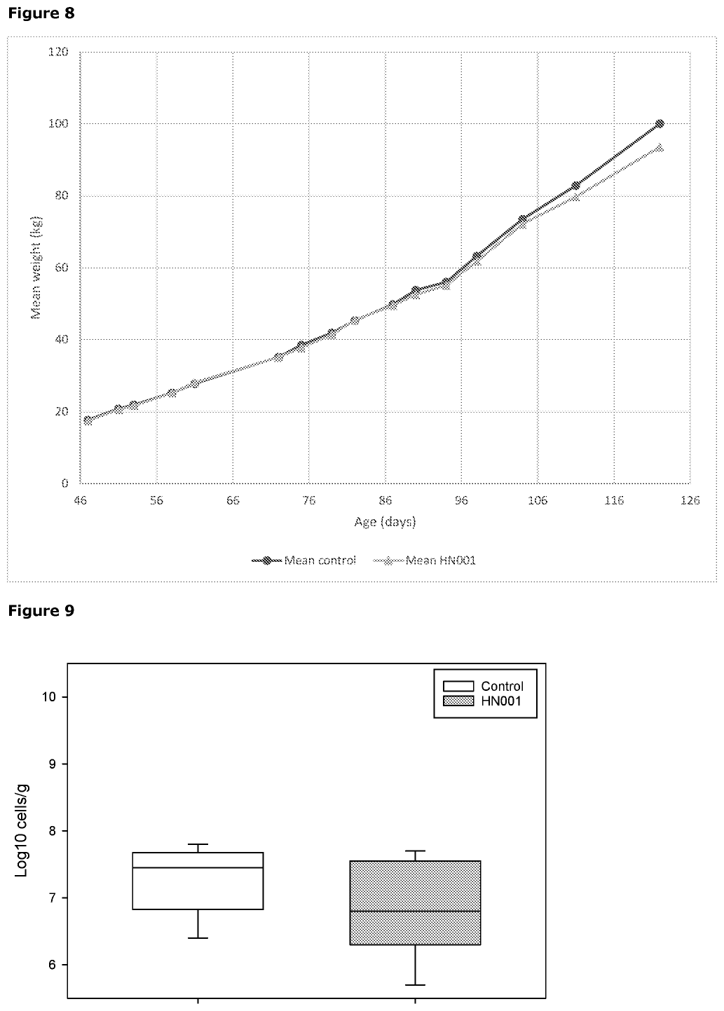

FIG. 8 show that supplementation with the bacterial strain HN001™ had no significant negative effect on pig growth and weight gain.

### 4.3 Conclusions

Supplementation of feed with L. rhamnosus HN001™ did not have a significant negative effect on pig weight. This work indicates that supplementation with HN001™ can reduce methane emissions without having a significant negative impact on weight gain.

### Example 5—The effect of Lacticaseibacillus rhamnosus HN001 on Methanogenic Bacteria in Pigs

### 5.1 Materials and Methods

### 5.1.1 Animal Trial: Experimental Design and Animal Ethics Approval

The piglets used in Example 4 were used for this trial.

### 5.1.2 Pig Gut Sample Collection

At 19 weeks of age, the pigs were euthanized (captive bolt stunning, weighed, followed by exsanguination), and their caecum and colorectal regions were tied off and removed to collect their digestive contents from these two gut regions. The gut contents samples were used for enumeration of methanogens using a Most Probable Number (MPN) method, and the remaining samples were for volatile fatty acid (VFA) analyses by Gas Chromatography. For the MPN analysis, 5 mL Eppendorfs were filled with caecal or colorectal contents for each animal and placed on ice until further processing in the laboratory. For VFA analysis, caecal contents were aliquoted into 50 mL Falcon tubes, while colorectal contents were sampled into 15 mL Falcon tubes, and placed immediately on ice prior to storage at −20° C.

### 5.1.3 Most Probable Number (MPN)

The MPN method (McCrady, 1918) was used to estimate the number of microorganisms able to produce methane from samples of caecal and colorectal contents. Briefly, measured amounts of sample (approximatively 1 g), were added into the first RM02 (Kenters et al. 2011) dilution tubes and the weights were recorded for use in the final calculations. From the first dilution tube, 10-fold serial dilutions (1 mL into 9 mL RM02 medium) were performed. Each dilution was mixed evenly, then further diluted a total of 10 times, under anaerobic conditions. Dilutions were selected from the series and samples were inoculated into Balch tubes containing BRN-RF10 medium (Balch et al., 1979; Hoedt, 2017) supplemented with methanol (100 mM final conc.) and pressurized with 180 kPa overpressure of H₂+CO₂. The inoculated BRN-RF10 medium tubes were incubated horizontally at 39° C. for 1 month. These conditions allow the growth of hydrogenotrophic organisms such as the methanogenic archaea and homoacetogens, as well as methylotrophic methanogens. After 1 month, the tubes were placed at room temperature for 30 min before carrying out headspace gas analysis using gas chromatography. Gas samples (0.5 mL) were collected from the tube headspace at the pressure in the culture vessel using polycarbonate 1 mL Luer-Lok syringes (Becton Dickinson and Co., Franklin Lakes, NJ, USA) fitted with Mininert Luer-tip syringe valves (Hamilton, Reno, NV, USA). The headspace gas samples were manually injected into an Aerograph 660 gas chromatograph (Varian Associates, Palo Alto, CA, USA) fitted with a Porapak Q80/100 mesh column (Waters Corporation, Milford, MA, USA) and a thermal conductivity detector. N₂ was used as the carrier gas. A 0.5 mL sample of gas standard containing H₂:CH₄:N₂ (5%:30%:65% v/v; BOC Gas, Palmerston North, NZ) was measured at 1 atm and used for calibration. The presence of methane was used as an indicator of methanogenic activity in the tube and was considered positive. If no methane was detected in the headspace, the tubes was considered as negative. By measuring the presence or absence of methane into the gaseous head space of the culture tubes, it is possible to determine in which tubes methanogens are present and metabolically active. From this data, and using MPN tables, the total number of methane-producing organism present in the original sample was calculated.

### 5.1.4 Media Preparation

RM02 was prepared anaerobically and dispensed under anaerobic conditions into Hungate tubes (9 mL per tube), then autoclaved for 20 min at 125° C. BRN-RF10 medium supplemented with (final concentrations) 60 mM sodium formate was prepared and dispensed under anaerobic condition into Balch tubes 9.8 mL per tube), then autoclaved for 20 min at 125° C. Before inoculation, 0.5% methanol (100 mM final), and 0.1 mL of Coenzyme M Solution (10 μM) were added by syringe using anaerobic techniques. After inoculation, the tubes were pressurised with 180 kPa over-pressure of H₂+CO₂ (80:20, BOC Gases NZ).

### 5.2 Results

TABLE 4
Most probable number (MPN) of methanogenic
bacteria per gram of pig faeces.

| | Caecum | | Colorectal | |
|---|---|---|---|---|
| | MPN | Fold change | MPN | Fold change |
| Control | 2.9 × 10⁷ | N/A | 4.8 × 10⁸ | N/A |
| HN001 ™ | 1.6 × 10⁷ | 1.70 | 2.4 × 10⁸ | 2.05 |

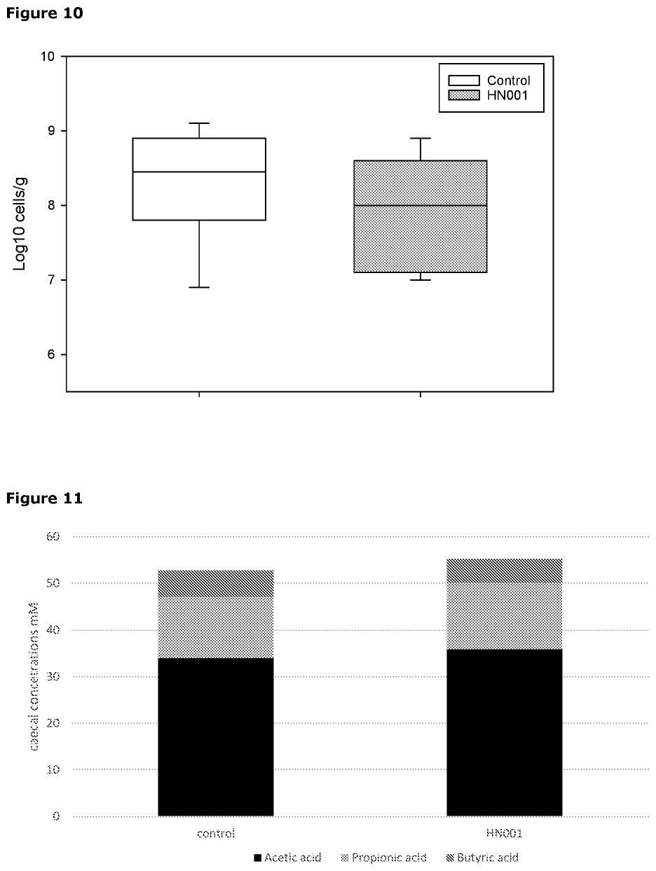

Table 4 and FIGS. 9 and 10 show a decrease in methanogen numbers and methanogenic activity in both the caecal and colorectal samples of pigs treated with HN001™.

### 5.3 Discussion

The pig intestinal microbiome shares many similarities with that of humans, including the dominance of the two major phyla the Bacteroidetes and Firmicutes, which occur in similar proportions in both species. While relative abundances may vary, bacteria associated with human health, such as Lactobacillus, Bifidobacterium, and Faecalibacterium are also commonly found in the pig. These similarities in the microbiome are likely to be a driven by the similarity in the digestive systems, which lead to common ecological constraints.

The MPN method allowed us to identify an impact of HN001 on caecal and colorectal methanogens. The feeding of the HN001 strain decreased the population level of methane-producing organisms in the pig caecum and colorectum, supporting the hypothesis that the strain could produce biological compound(s) inhibiting the growth of methanogens.

Independently of treatment, the lower methanogen populations in the pig caecum compared to the colorectum was expected as methanogens in monogastric animals prefer the terminal part of the digestive tract. Methanogen communities, as estimated by copy numbers of 16S rRNA genes, range from ˜10⁸ to 10⁹ organisms per gram of digestive contents from caecum to rectum. Similarly, methane percentage in gases produced from the gut range from 1.7-2.5% to 29-38%, from the caecum to the rectum, respectively. An increasing gradient of methanogen populations and methane formation in the pig hindgut from the caecum to the rectum exists (Jorgensen et 2011, Gresse et al 2019) and reflects slower transit times, more anaerobic conditions, and higher pH from the caecum to the colorectum. These conditions favour methanogens, allowing them to replicate and maintain their populations more easily.

Probiotics administered to pigs will first encounter methanogens after they pass through the stomach and small intestine and enter the caecum. Because methanogen numbers and activity are lower in the caecum, the effect of the probiotic strains are likely to have their greatest anti-methanogen effect in this gut compartment. Moreover, it is also expected to see the impact of probiotics on microbial fermentation through altered VFA profiles in this region of the gut as the caecum represents a preferred niche for lactobacilli. The LAB are likely be most active in this region and interact with other member of the ecosystem, or produce and/or release compounds into the caecum, to have their inhibitory effect on methanogen populations.

### 5.4 Conclusions

This Example shows that feed supplementation with L. rhamnosus HN001™ can reduce the amount of methanogenic microorganisms in the gut of pigs.

### 6. Example 6—The Effect of Lacticaseibacillus rhamnosus HN001 on Volatile Fatty Acid Production in Pigs

### 6.1 Materials and Methods

### 6.1.1 Sample Collection

The pigs used in Examples 4 and 5 were used in this experiment. Directly after euthanasia, the pig caecum and colorectum gut regions were tied off and removed. Caecal contents were aliquoted into 50 mL Falcon tubes, while colorectal contents were sampled into 15 mL Falcon tubes, and placed immediately onto ice to be transferred into a −20° C. freezer until further analysis.

### 6.1.2 Sample Preparation for Gas Chromatography Analysis of VFAs

Samples were defrosted at room temperature. An aliquot (0.5 mL) of each sample was first removed. The remaining sample volume was centrifuged at 21,000×g for 10 min at 4° C. and 0.9 mL of the supernatant was removed and added to 0.1 mL of internal standard (20 mM 2-ethylbutyrate in 20% phosphoric acid), mixed and frozen at −20° C. until analysis. After thawing and re-centrifugation at 21,000×g for 10 min at 4° C., 0.2 mL of the supernatant was collected for derivatization for non-VFA analysis using gas chromatography (Richardson et al. 1989), while the remainder of the sample was analysed directly via gas chromatography (Attwood et al., 1998), using a gas chromatograph (Model 6869, Hewlett-Packard, Montreal, QC, Canada) equipped with an auto-sampler, and fitted with a Zebron ZB-FFAP 30.0 m×0.53 mm I.D.×1 μm film column (Phenomenex, Torrance, CA, United States) and a flame ionization detector set at 265° C.

### 6.2 Results

The main VFAs present in the caecal and colorectal sample were acetic, propionic, and butyric acids. Their respective concentrations and proportions correspond to normal levels of VFA present in monogastric animals.

### 6.2.1 Caecal VFAs

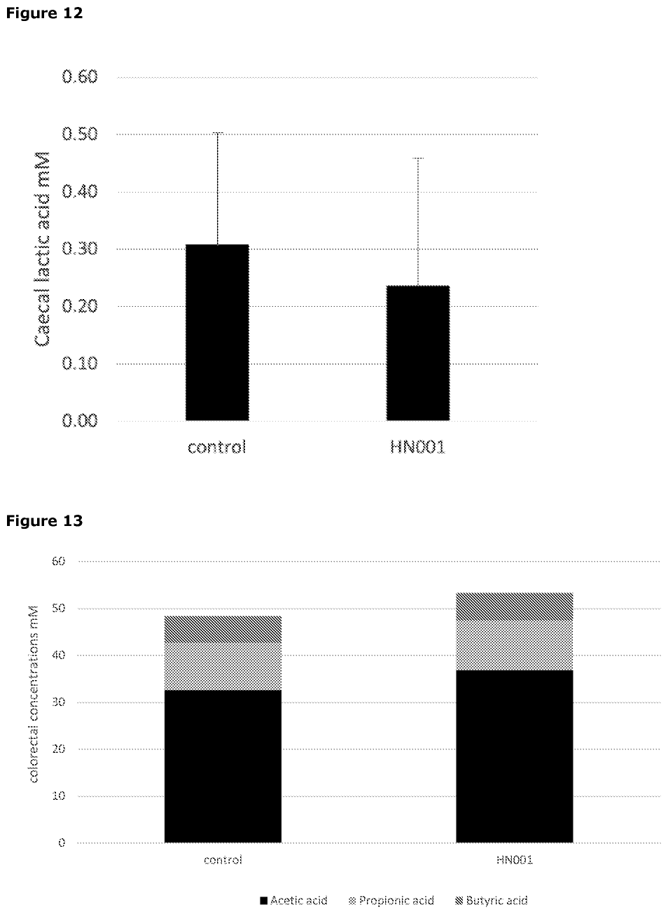

No impact of HN001 on caecal VFA concentrations/proportions was observed (Table 5 and FIGS. 11 and 12).

TABLE 5
Main volatile fatty acid percentages in pig caecal samples
collected after euthanasia. n = 8 animals per group.

| | % Acetic acid | % Propionic acid | % Butyric acid |
|---|---|---|---|
| Control | 64 ± 3 | 24 ± 3 | 10 ± 3 |
| HN001 ™ | 64 ± 4 | 26 ± 4 | 9 ± 2 |

### 6.2.2 Colorectal VFAs

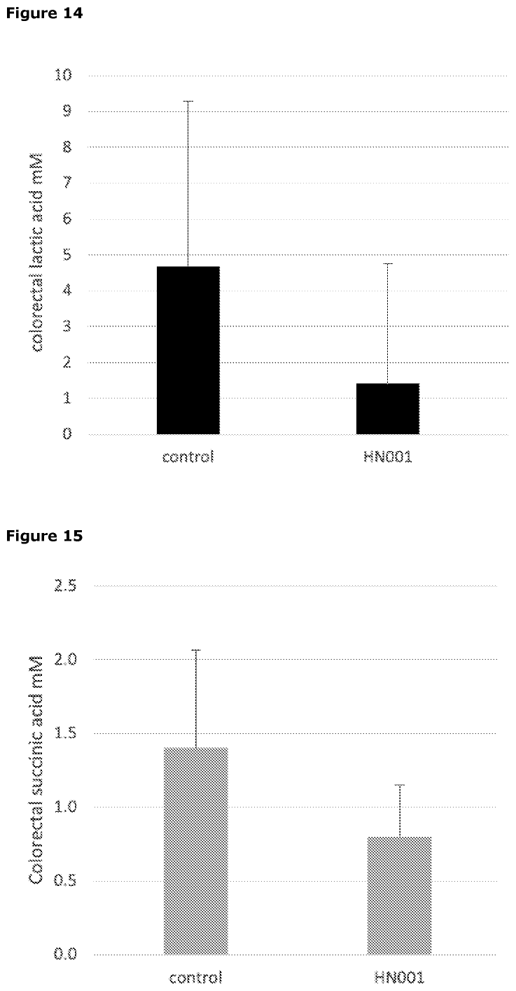

The results indicated that there was no impact of L. rhamnosus HN001™ on the colorectal fermentation process (Table 6 and FIGS. 13 to 15). The proportion of acetic acid in colorectal samples was higher than the control, however the difference was not significant.

TABLE 6
Main volatile fatty acid percentages in pig colorectal samples
collected after euthanasia. n = 8 animals per group.

| | % Acetic acid | % Propionic acid | % Butyric acid |
|---|---|---|---|
| Control | 55 ± 7 | 17 ± 1 | 9 ± 2 |
| HN001 ™ | 61 ± 6 | 18 ± 2 | 9 ± 2 |

### 6.3 Conclusions

This example shows that the specific inhibitory effect on methanogens, and the reduction in the amount of methanogenic microorganisms in the gut of pigs, following feed supplementation with L. rhamnosus HN001™ shown in the earlier Examples can occur without significantly altering the volatile fatty acid production, live weight, or average daily gain of the animals.

### 7. Example 7—On-Farm Cell Culture

### 7.1 Materials and Methods

A mixture of cultures of L. rhamnosus HN001™ and Lactobacillus lactis subsp. cremoris 2566 were added to thermalised milk with and without yeast extract (YE) and incubated using a water bath held at 25° C. or 30° C. for 12 hours. Viable cell counts were measured.

### 7.2 Results

L. rhamnosus HN001™ was able to grow well in a thermalised milk medium at either 25° C. or 30° C., achieving viable cell counts in excess of 5×10⁸ cells/g in combined culture with L. lactis subsp. cremoris 2566 (Table 15). The addition of yeast extract (YE) slightly increased the viable cell count of L. rhamnosus HN001™.

TABLE 15
Viable cell counts.

Viable cell count (cells/g)

| | L. rhamnosus | L. lactis subsp. |
|---|---|---|
| Treatment | HN001 | cremoris 2566 |
| 30° C. + YE | 1.95 × 10⁸ | 9.50 × 10⁸ |
| 30° C. | 1.70 × 10⁸ | 1.11 × 10⁹ |
| 25° C. + YE | 5.10 × 10⁸ | 1.35 × 10⁹ |
| 25° C. | 9.50 × 10⁷ | 9.10 × 10⁸ |

### 7.3 Conclusion

This Example shows that L. rhamnosus HN001™ can be cultured to high cell density using a thermalised milk medium, suitable for on-farm applications.

Preferred embodiments of the invention have been described by way of example only and modifications may be made thereto without departing from the scope of the invention.

### REFERENCES

Anderson, R. C., Cookson, A. L., McNabb, W. C., Kelly, W. J., Roy, N. C., 2010. Lactobacillus plantarum DSM 2648 is a potential probiotic that enhances intestinal barrier function. FEMS Microbiol. Lett. 309, 184-192. https://doi.org/10.1111/j.1574-6968.2010.02038.x

Attwood, G. T., A. V. Klieve, D. Ouwerkerk, and B. K. C Patel. 1998. Ammonia-hyper producing bacteria from New Zealand ruminants. Applied and Environmental Microbiology, 64:1796-1804.

Balch, W. E., Fox, G. E., Magrum, L. J., Woese, C. R. & Wolfe, R. S. Methanogens: re-evaluation of a unique biological group. Microbiological Reviews, 1979; 43, 260-96.

Bengtsson-Palme, J., Hartmann, M., Eriksson, K. M., Pal, C., Thorell, K., Larsson, D. G. J., Nilsson, R. H., 2015. METAXA2: improved identification and taxonomic classification of small and large subunit rRNA in metagenomic data. Mol. Ecol. Resour. 15, 1403-1414. https://doi.org/10.1111/1755-0998.12399

Bolger, A. M., Lohse, M., Usadel, B., 2014. Trimmomatic: a flexible trimmer for Illumina sequence data. Bioinforma. Oxf. Engl. 30, 2114-2120. https://doi.org/10.1093/bioinformatics/btu170

Buchfink, B., Xie, C., Huson, D. H., 2015. Fast and sensitive protein alignment using DIAMOND. Nat. Methods 12, 59-60. https://doi.org/10.1038/nmeth.3176

Bushnell, B., 2014. BBMap: A Fast, Accurate, Splice-Aware Aligner (No. LBNL-7065E). Lawrence Berkeley National Lab. (LBNL), Berkeley, CA (United States).

Carbonero, F., Benefiel, A. C., Gaskins, H. R., 2012. Contributions of the microbial hydrogen economy to colonic homeostasis. Nat. Rev. Gastroenterol. Hepatol. 9, 504-518. https://doi.org/10.1038/nrgastro.2012.85

Carlson, M., 2016. KEGG.db: A set of annotation maps for KEGG.

De Filippis, F., Pasolli, E., Tett, A., Tarallo, S., Naccarati, A., De Angelis, M., Neviani, E., Cocolin, L., Gobbetti, M., Segata, N., Ercolini, D., 2019. Distinct Genetic and Functional Traits of Human Intestinal Prevotella copri Strains Are Associated with Different Habitual Diets. Cell Host Microbe 25, 444-453.e3. https://doi.org/10.1016/j.chom.2019.01.004

Dixon, P., 2003. VEGAN, a package of R functions for community ecology. J. Veg. Sci. 14, 927-930. https://doi.org/10.1111/j.1654-1103.2003.tb02228.x

Dobin, A., Davis, C. A., Schlesinger, F., Drenkow, J., Zaleski, C., Jha, S., Batut, P., Chaisson, M., Gingeras, T. R., 2013. STAR: ultrafast universal RNA-seq aligner. Bioinforma. Oxf. Engl. 29, 15-21. https://doi.org/10.1093/bioinformatics/bts635

Doyle, N., Mbandlwa, P., Kelly, W. J., Attwood, G., Li, Y., Ross, R. P., Stanton, C., Leahy, S., 2019. Use of Lactic Acid Bacteria to Reduce Methane Production in Ruminants, a Critical Review. Front. Microbiol. 10. https://doi.org/10.3389/fmicb.2019.02207

Efimov, B. A., Chaplin, A. V., Shcherbakova, V. A., Suzina, N. E., Podoprigora, I. V., Shkoporov, A. N., 2018. Prevotella rara sp. nov., isolated from human faeces. Int. J. Syst. Evol. Microbiol. 68, 3818-3825. https://doi.org/10.1099/ijsem.0.003066

Filippo, C. D., Cavalieri, D., Paola, M. D., Ramazzotti, M., Poullet, J. B., Massart, S., Collini, S., Pieraccini, G., Lionetti, P., 2010. Impact of diet in shaping gut microbiota revealed by a comparative study in children from Europe and rural Africa. Proc. Natl. Acad. Sci. 107, 14691-14696. https://doi.org/10.1073/pnas.1005963107

Flint, H. J., Scott, K. P., Duncan, S. H., Louis, P., Forano, E., 2012. Microbial degradation of complex carbohydrates in the gut. Gut Microbes 3, 289-306. https://doi.org/10.4161/gmic.19897

Good, M., Sodhi, C. P., Ozolek, J. A., Buck, R. H., Goehring, K. C., Thomas, D. L., Vikram, A., Bibby, K., Morowitz, M. J., Firek, B., Lu, P., Hackam, D. J., 2014. Lactobacillus rhamnosus HN001 decreases the severity of necrotizing enterocolitis in neonatal mice and preterm piglets: evidence in mice for a role of TLR9. Am. J. Physiol. Gastrointest. Liver Physiol. 306, G1021-1032. https://doi.org/10.1152/ajpgi.00452.2013

Gresse, R., Durand, F. C., Dunière, L., Blanquet-Diot, S., Forano, E., 2019. Microbiota Composition and Functional Profiling Throughout the Gastrointestinal Tract of Commercial Weaning Piglets. Microorganisms 7(9), 343. https://doi.org/10.3390/microorganisms7090343

Groschwitz, K. R., Hogan, S. P., 2009. Intestinal barrier function: molecular regulation and disease pathogenesis. J. Allergy Clin. Immunol. 124, 3-20; quiz 21-22. https://doi.org/10.1016/j.jaci.2009.05.038

Hoedt, E. C. 2017. Functional and comparative studies of members of the genus Methanosphaera, and their adaptations to the gut environment. PhD Thesis, The University of Queensland, Brisbane, Australia. https://doi.org/10.14264uql.2017.366

Hjorth, M. F., Blaedel, T., Bendtsen, L. Q., Lorenzen, J. K., Holm, J. B., Kiilerich, P., Roager, H. M., Kristiansen, K., Larsen, L. H., Astrup, A., 2019. Prevotella-to-Bacteroides ratio predicts body weight and fat loss success on 24-week diets varying in macronutrient composition and dietary fiber: results from a post-hoc analysis. Int. J. Obes. 2005 43, 149-157. https://doi.org/10.1038/s41366-018-0093-2

Huang, L., Zhang, D., Han, W., Guo, C., 2019. High-mobility group box-1 inhibition stabilizes intestinal permeability through tight junctions in experimental acute necrotizing pancreatitis. Inflamm. Res. Off. J. Eur. Histamine Res. Soc. Al 68, 677-689. https://doi.org/10.1007/s00011-019-01251-x

Huson, D. H., Beier, S., Flade, I., Górska, A., El-Hadidi, M., Mitra, S., Ruscheweyh, H.-J., Tappu, R., 2016. MEGAN Community Edition—Interactive Exploration and Analysis of Large-Scale Microbiome Sequencing Data. PLOS Comput. Biol. 12, e1004957. https://doi.org/10.1371/journal.pcbi.1004957

Iljazovic, A., Roy, U., Gálvez, E. J. C., Lesker, T. R., Zhao, B., Gronow, A., Amend, L., Will, S. E., Hofmann, J. D., Pils, M. C., Schmidt-Hohagen, K., Neumann-Schaal, M., Strowig, T., 2020. Perturbation of the gut microbiome by Prevotella spp. enhances host susceptibility to mucosal inflammation. Mucosal Immunol. 1-12. https://doi.org/10.1038/s41385-020-0296-4

IPCC, 2014. Mitigation of Climate Change. Contribution of Working Group III to the Fifth Assessment Report of the Intergovernmental Panel on Climate Change. Cambridge University Press, Cambridge.

Jackson, R. B., Saunois, M., Bousquet, P., Canadell, J. G., Poulter, B., Stavert, A. R., Bergamaschi, P., Niwa, Y., Segers, A., Tsuruta, A., 2020. Increasing anthropogenic methane emissions arise equally from agricultural and fossil fuel sources. Environ. Res. Lett. 15, 071002. https://doi.org/10.1088/1748-9326/ab9ed2

Johnson, J. S., Spakowicz, D. J., Hong, B.-Y., Petersen, L. M., Demkowicz, P., Chen, L., Leopold, S. R., Hanson, B. M., Agresta, H. O., Gerstein, M., Sodergren, E., Weinstock, G. M., 2019. Evaluation of 16S rRNA gene sequencing for species and strain-level microbiome analysis. Nat. Commun. 10, 5029. https://doi.org/10.10381/s41467-019-13036-1

Jørgensen, H., Theil, P. K., Knudsen, K. E. B. Enteric methane emission from pigs. Planet Earth 2011—Global Warming Challenges and Opportunities for Policy and Practice, 605-622.

Kanehisa, M., Goto, S., 2000. KEGG: kyoto encyclopedia of genes and genomes. Nucleic Acids Res. 28, 27-30. https://doi.org/10.1093/nar/28.1.27

Kawasaki, T., Kawai, T., 2014. Toll-Like Receptor Signaling Pathways. Front. Immunol. 5. https://doi.org/10.3389/fiimmu.2014.00461

Kenters N, Henderson G, Jeyanathan J, Kittelmann S, Janssen P H. Isolation of previously uncultured rumen bacteria by dilution to extinction using a new liquid culture medium. J Microbiol Methods. 2011 January; 84(1):52-60.

Kovatcheva-Datchary, P., Nilsson, A., Akrami, R., Lee, Y. S., De Vadder, F., Arora, T., Hallen, A., Martens, E., Björck, I., Bäckhed, F., 2015. Dietary Fiber-Induced Improvement in Glucose Metabolism Is Associated with Increased Abundance of Prevotella. Cell Metab. 22, 971-982. https://doi.org/10.1016/j.cmet.2015.10.001

LeBlanc, J. G., Laiño, J. E., del Valle, M. J., Vannini, V., van Sinderen, D., Taranto, M. P., de Valdez, G. F., de Giori, G. S., Sesma, F., 2011. B-group vitamin production by lactic acid bacteria-current knowledge and potential applications. J. Appl. Microbiol. 111, 1297-1309. https://doi.org/10.1111/j.1365-2672.2011.05157.x

Leite, A. Z., Rodrigues, N. de C., Gonzaga, M. I., Paiolo, J. C. C., de Souza, C. A., Stefanutto, N. A. V., Omori, W. P., Pinheiro, D. G., Brisotti, Matheucci Junior, E., Mariano, V. S., de Oliveira, G. L. V., 2017. Detection of Increased Plasma Interleukin-6 Levels and Prevalence of Prevotella copri and Bacteroides vulgatus in the Feces of Type 2 Diabetes Patients. Front. Immunol. 8, 1107. https://doi.org/10.3389/fimmu.2017.01107

Loughman, A., Ponsonby, A.-L., O'Hely, M., Symeonides, C., Collier, F., Tang, M. L. K., Carlin, J., Ranganathan, S., Allen, K., Pezic, A., Saffery, R., Jacka, F., Harrison, L. C., Sly, P. D., Vuillermin, P., 2020. Gut microbiota composition during infancy and subsequent behavioural outcomes. EBioMedicine 52. https://doi.org/10.1016/j.ebiom.2020.102640

Matsuzawa-Ishimoto, Y., Hwang, S., Cadwell, K., 2018. Autophagy and Inflammation. Annu. Rev. Immunol. 36, 73-101. https://doi.org/10.1146/annurev-immunol-042617-053253

McCrady, M. H., 1918. Tables for Rapid Interpretation of Fermentation-tube Results. The Public Health Journal, n9(5): 201-220.

Mi, J., Peng, H., Wu, Y., Wang, Y., & Liao, X., 2019. Diversity and community of methanogens in the large intestine of finishing pigs. BMC Microbiology 19:83.

Ursula M. McCormack, Tânia Curiã, Barbara U. Metzler-Zebeli, Elizabeth Magowan, Donagh P. Berry, Henry Reyer, Maria L. Prieto, Stefan G. Buzoianu, Michael Harrison, Natalie Rebeiz, Fiona Crispie, Paul D. Cotter, Orla O'Sullivan, Gillian E. Gardiner, Peadar G. Lawlor (2019). Porcine Feed Efficiency-Associated Intestinal Microbiota and Physiological Traits: Finding Consistent Cross-Locational Biomarkers for Residual Feed Intake. mSystems 4 (4):e00324-18; DOI: 10.1128/mSystems.00324-18.

A. Misiukiewicz, M. Gao, W. Filipiak, A. Cieslak, A. K. Patra, M. Szumacher-Strabel, 2021. Review: Methanogens and methane production in the digestive systems of nonruminant farm animals, Animal, Volume 15, Issue 1. https://doi.org/10.1016/j.animal.2020.100060 Morishita, T., Tamura, N., Makino, T., Kudo, S., 1999. Production of menaquinones by lactic acid bacteria. J. Dairy Sci. 82, 1897-1903. https://doi.org/10.3168/jds.S0022-0302(99)75424-X

Mudd, A. T., Fleming, S. A., Labhart, B., Chichlowski, M., Berg, B. M., Donovan, S. M., Dilger, R. N., 2017. Dietary Sialyllactose Influences Sialic Acid Concentrations in the Prefrontal Cortex and Magnetic Resonance Imaging Measures in Corpus Callosum of Young Pigs. Nutrients 9. https://doi.org/10.3390/nu9121297

Nikoletopoulou, V., Sidiropoulou, K., Kallergi, E., Dalezios, Y., Tavernarakis, N., 2017. Modulation of Autophagy by BDNF Underlies Synaptic Plasticity. Cell Metab. 26, 230-242.e5. https://doi.org/10.1016/j.cmet.2017.06.005

Overbeek, R., Begley, T., Butler, R. M., Choudhuri, J. V., Chuang, H.-Y., Cohoon, M., de Crécy-Lagard, V., Diaz, N., Disz, T., Edwards, R., Fonstein, M., Frank, E. D., Gerdes, S., Glass, E. M., Goesmann, A., Hanson, A., Iwata-Reuyl, D., Jensen, R., Jamshidi, N., Krause, L., Kubal, M., Larsen, N., Linke, B., McHardy, A. C., Meyer, F., Neuweger, H., Olsen, G., Olson, R., Osterman, A., Portnoy, V., Pusch, G. D., Rodionov, D. A., Rückert, C., Steiner, J., Stevens, R., Thiele, I., Vassieva, O., Ye, Y., Zagnitko, O., Vonstein, V., 2005. The subsystems approach to genome annotation and its use in the project to annotate 1000 genomes. Nucleic Acids Res. 33, 5691-5702. https://doi.org/10.1093/nar/gki866

Peabody, M. A., Van Rossum, T., Lo, R., Brinkman, F. S. L., 2015. Evaluation of shotgun metagenomics sequence classification methods using in silico and in vitro simulated communities. BMC Bioinformatics 16, 362. https://doi.org/10.1186/s12859-015-0788-5

Precup, G., Vodnar, D.-C., 2019. Gut Prevotella as a possible biomarker of diet and its eubiotic versus dysbiotic roles: a comprehensive literature review. Br. J. Nutr. 122, 131-140. https://doi.org/10.1017/S0007114519000680

Quast, C., Pruesse, E., Yilmaz, P., Gerken, J., Schweer, T., Yarza, P., Peplies, J., Glöckner, F. O., 2013. The SILVA ribosomal RNA gene database project: improved data processing and web-based tools. Nucleic Acids Res. 41, D590-D596. https://doi.org/10.1093/nar/gks1219

Reinshagen, M., Rohm, H., Steinkamp, M., Lieb, K., Geerling, I., Von Herbay, A., Flämig, G., Eysselein, V. E., Adler, G., 2000. Protective role of neurotrophins in experimental inflammation of the rat gut. Gastroenterology 119, 368-376. https://doi.org/10.1053/gast.2000.9307

Riboulet-Bisson, E., Sturme, M. H. J., Jeffery, I. B., O'Donnell, M. M., Neville, B. A., Forde, B. M., Claesson, M. J., Harris, H., Gardiner, G. E., Casey, P. G., Lawlor, P. G., O'Toole, P. W., Ross, R. P., 2012. Effect of Lactobacillus salivarius bacteriocin Abp118 on the mouse and pig intestinal microbiota. PloS One 7, e31113. https://doi.org/10.1371/journal.pone.0031113

Richardson, A. J., Calder, A. G., Stewart, C., & Smith, A. Simultaneous determination of volatile and non-volatile acidic fermentation products of anaerobes by capillary gas chromatography. Letters in Applied Microbiology; March 2008; 9(1):5-8.

Robinson, M. D., McCarthy, D. J., Smyth, G. K., 2010. edgeR: a Bioconductor package for differential expression analysis of digital gene expression data. Bioinforma. Oxf. Engl. 26, 139-140. https://doi.org/10.1093/bioinformatics/btp616

Roux, V., Robert, C., Raoult, D., 2014. Non-contiguous finished genome sequence of Prevotella timonensis type strain 4401737(T.). Stand. Genomic Sci. 9, 1344-1351. https://doi.org/10.4056/sigs.5098948

Scher, J. U., Sczesnak, A., Longman, R. S., Segata, N., Ubeda, C., Bielski, C., Rostron, T., Cerundolo, V., Pamer, E. G., Abramson, S. B., Huttenhower, C., Littman, D. R., 2013. Expansion of intestinal Prevotella copri correlates with enhanced susceptibility to arthritis. eLife 2, e01202. https://doi.org/10.7554/eLife.01202

Smith, N. W., Shorten, P. R., Altermann, E. H., Roy, N. C., McNabb, W. C., 2019. Hydrogen cross-feeders of the human gastrointestinal tract. Gut Microbes 10, 270-288. https://doi.org/10.1080/19490976.2018.1546522

Smyth, G. K., 2004. Linear models and empirical bayes methods for assessing differential expression in microarray experiments. Stat. Appl. Genet. Mol. Biol. 3, Article 3. https://doi.org/10.2202/1544-6115.1027

Soto-Martin, E. C., Warnke, I., Farquharson, F. M., Christodoulou, M., Horgan, G., Derrien, M., Faurie, J.-M., Flint, H. J., Duncan, S. H., Louis, P., 2020. Vitamin Biosynthesis by Human Gut Butyrate-Producing Bacteria and Cross-Feeding in Synthetic Microbial Communities. mBio 11. https://doi.org/10.1128/mBio.00886-20

Tyrrell, H. F., Reid, J. T., 1965. Prediction of the energy value of cow's milk. J. Dairy Sci. 48, 1215-1223. https://doi.org/10.3168/jds.S0022-0302(65)88430-2

Wheeler, B., Torchiano, M., 2016. ImPerm: Permutation tests for linear models.

Wong, M., Ganapathy, A. S., Suchanec, E., Laidler, L., Ma, T., Nighot, P., 2019. Intestinal epithelial tight junction barrier regulation by autophagy-related protein ATG6/beclin 1. Am. J. Physiol. Cell Physiol. 316, C753-C765. https://doi.org/10.1152/ajpcell.00246.2018

Wright, D. P., Rosendale, D. I., Robertson, A. M., 2000. Prevotella enzymes involved in mucin oligosaccharide degradation and evidence for a small operon of genes expressed during growth on mucin. FEMS Microbiol. Lett. 190, 73-79. https://doi.org/10.1111/j.1574-6968.2000.tb09265.x

Zhang, J., Kobert, K., Flouri, T., Stamatakis, A., 2014. PEAR: a fast and accurate Illumina Paired-End reAd mergeR. Bioinforma. Oxf. Engl. 30, 614-620. https://doi.org/10.1093/bioinformatics/btt593

### INDUSTRIAL APPLICABILITY

This invention relates to methods for inhibiting the growth of methane-producing bacteria and/or archaea in the gastrointestinal tract of monogastric animals and/or for reducing methane emissions by a monogastric animal, and/or for increasing feed efficiency, and/or body weight or body composition of a monogastric animal.

What we claim is:

1. A method for inhibiting the growth of methane-producing bacteria and/or archaea in the gastrointestinal tract of monogastric animals, wherein the method comprises administering to a monogastric animal an effective amount of Lacticaseibacillus rhamnosus strain HN001, AGAL deposit number NM97/09514 dated 18 Aug. 1997, or a derivative thereof.

2. A method for reducing methane production by a monogastric animal, wherein the method comprises administering to the animal an effective amount of Lacticaseibacillus rhamnosus strain HN001, AGAL deposit number NM97/09514 dated 18 Aug. 1997, or a derivative thereof.

3. A method for increasing feed efficiency in a monogastric animal, wherein the method comprises administering to the animal an effective amount of Lacticaseibacillus rhamnosus strain HN001, AGAL deposit number NM97/09514 dated 18 Aug. 1997, or a derivative thereof.

4. A method for reducing the ability of the microbiome of the gastrointestinal tract to produce methane, wherein the method comprises administering to the animal an effective amount of Lacticaseibacillus rhamnosus strain HN001, AGAL deposit number NM97/09514 dated 18 Aug. 1997, or a derivative thereof.

5. The method of any preceding claim, wherein the method inhibits the growth of hydrogenotrophic methanogens in the caecum of the animal, preferably a methanogen from the genus Methanobrevibacter.

6. The method of any preceding claim, wherein L. rhamnosus HN001 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, supplement, pharmaceutical, lick block, drench, tablet, capsule, pellet or bolus, or wherein the L. rhamnosus HN001 is encapsulated in liposomes, microbubbles, microparticles or microcapsules.

7. The method of claim 6, wherein L. rhamnosus HN001 or derivative thereof is administered in drinking water, milk, milk powder, milk replacement, milk fortifier, whey, whey powder, a feed pellet, corn, soybean, forage, grain, distiller's grain, sprouted grain, legumes, vitamins, amino acids, minerals, fibre, fodder, grass, hay, silage, kernel, leaves, meal, solubles, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushd limestone.

8. The method of any preceding claim, wherein the method comprises administering to the animal L. rhamnosus HN001 in an amount of from 10⁴ to 10¹³ colony forming units per kilogram of dry weight carrier feed, from 10⁴ to 10¹⁰ colony forming units per kilogram of body weight of the animal, or from 10⁴ to 10¹³ colony forming units.

9. The method of claim 8, wherein the method comprises administering to the animal L. rhamnosus HN001 in an amount from 10⁸ to 10¹² colony forming units per kilogram of dry weight carrier feed, from 10⁵ to 10⁸ colony forming units per kilogram of body weight of the animal, or from 10⁶ to 10¹³ colony forming units.

10. The method of any preceding claim, wherein the derivative of L. rhamnosus HN001 is a cell lysate of the strain, a cell suspension of the strain, a metabolite of the strain, a culture supernatant of the strain, or killed L. rhamnosus HN001.

11. The method of any preceding claim, the method comprising further administering at least one additional microorganism of a different species or strain, a vaccine that inhibits methanogens or methanogenesis, and/or a natural or chemically-synthesised inhibitor of methanogenesis and/or methanogen inhibitor, such as bromoform.

12. The method of any preceding claim, wherein the L. rhamnosus HN001 or derivative thereof is administered separately, simultaneously or sequentially with one or more agents selected from one or more prebiotics, one or more probiotics, one or most postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

13. The method of any preceding claim, wherein the method additionally improves the body weight and/or body composition of the monogastric animal.

14. The method of any preceding claim, wherein said monogastric animal is a human, pig, cat, dog, horse, donkey, rabbit, or poultry.

15. The method of any preceding claim, wherein said monogastric animal is a companion animal.

16. The method of any preceding claim, wherein said monogastric animal is a non-human animal.

17. The method of any one of claims 1 to 14, wherein said monogastric animal is a pig.

18. The method of any one of claims 1 to 14, wherein said monogastric animal is a chicken, duck, goose or turkey.

19. The method of any one of claims 1 to 15, wherein said monogastric animal is a pre-weaning animal, for example a piglet or a foal.

20. The method of any one of claims 1 to 15, wherein said monogastric animal is a post-weaning animal.

21. The method of any one of claims 1 to 15, wherein the L. rhamnosus HN001 is administered to the monogastric animal both prior to weaning and after weaning.

22. The method of any one of claims 1 to 15, wherein the administering is to a pre-weaning animal and wherein the inhibiting the growth of methane-producing bacteria and/or archaea in the gastrointestinal tract of monogastric animals, the reducing the ability of the microbiome of the gastrointestinal tract to produce methane, the reducing methane production by the monogastric animal, and/or the increasing feed efficiency in the monogastric animal persists post-weaning.

23. The method of claim 22, wherein the inhibiting the growth of methane-producing bacteria and/or archaea in the gastrointestinal tract of monogastric animals, the reducing the ability of the microbiome of the gastrointestinal tract to produce methane, the reducing methane production by the monogastric animal, and/or the increasing feed efficiency in the monogastric animal persists for at least 2 days, 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus HN001.

24. The method of claim 23, wherein the inhibiting the growth of methane-producing bacteria and/or archaea in the gastrointestinal tract of monogastric animals, the reducing the ability of the microbiome of the gastrointestinal tract to produce methane, the reducing methane production by the monogastric animal, and/or the increasing feed efficiency in the monogastric animal persists for the life of the monogastric animal.

25. The method of any previous claim, wherein the L. rhamnosus HN001 is administered in a composition that is a fermented yoghurt-style composition, and wherein the fermented yoghurt-style composition is formed through a process of growing L. rhamnosus HN001 using a milk-based carrier.

26. A method for enhancing the growth and/or productivity in a monogastric animal, wherein the method comprises administering to a monogastric animal an effective amount of Lacticaseibacillus rhamnosus strain HN001, AGAL deposit number NM97/09514 dated 18 Aug. 1997, or a derivative thereof.

27. A method for improving the body weight and/or body composition of monogastric animal, wherein the method comprises administering to a monogastric animal an effective amount of Lacticaseibacillus rhamnosus strain HN001, AGAL deposit number NM97/09514 dated 18 Aug. 1997, or a derivative thereof.

28. Use of Lacticaseibacillus rhamnosus strain HN001, AGAL deposit number NM97/09514 dated 18 Aug. 1997, or a derivative thereof for the manufacture of a composition for inhibiting the growth of methane-producing bacteria and/or archaea in the gastrointestinal tract of monogastric animals, reducing the ability of the microbiome of the gastrointestinal tract to produce methane, reducing methane production by a monogastric animal, increasing feed efficiency in a monogastric animal, or improving the body weight and/or body composition of a monogastric animal.

29. The use of claim 28, wherein the composition is a medicament.

30. The use of claim 28 or 29, wherein the monogastric animal is a human.

31. Lacticaseibacillus rhamnosus strain HN001, AGAL deposit number NM97/09514 dated 18 Aug. 1997, or a derivative thereof for use in inhibiting the growth of methane-producing bacteria and/or archaea in the gastrointestinal tract of monogastric animals, reducing the ability of the microbiome of the gastrointestinal tract to produce methane, reducing methane production by a monogastric animal, increasing feed efficiency in a monogastric animal, or improving the body weight and/or body composition of a monogastric animal.

32. The L. rhamnosus HN001 or derivative thereof for use of claim 31, wherein the monogastric animal is a human.

33. Use of Lacticaseibacillus rhamnosus strain HN001, AGAL deposit number NM97/09514 dated 18 Aug. 1997, or a derivative thereof, for inhibiting the growth of methane-producing bacteria and/or archaea in the gastrointestinal tract of monogastric animals, reducing the ability of the microbiome of the gastrointestinal tract to produce methane, reducing methane production by a monogastric animal, increasing feed efficiency in a monogastric animal, or improving the body weight and/or body composition of a monogastric animal.

34. Use of a composition comprising Lacticaseibacillus rhamnosus strain HN001, AGAL deposit number NM97/09514 dated 18 Aug. 1997, or a derivative thereof, for inhibiting the growth of methane-producing bacteria and/or archaea in the gastrointestinal tract of monogastric animals, reducing the ability of the microbiome of the gastrointestinal tract to produce methane, reducing methane production by a monogastric animal, increasing feed efficiency in a monogastric animal, or improving the body weight and/or body composition of a monogastric animal.

35. The use of claim 33 or 34, wherein the monogastric animal is a human.
